SEQUENCE LISTING (26 sequences)

SEQ ID NO: 1 - protein, 760 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTENQVSLTCLVKGFYPSDIAVEWESNGQPENNYKTTPPVLDSDGSFFLYSWLTVDKSRWQEGNVFSCSVMHEALHNHYTQKSLSLSLGKAAAEGKSSGSGSESKSTSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGGSGGSGGSGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 557 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPRVYTLPPCQEEMTKNQVSLTCLVKGFYPSDIAVEWESNGQPENNYKTTPPVLVSDGSFTLYSRLTVDKSRWQEGNVFSCSVMHEALHNHYTQKSLSLSLGKAAAEGKSSGSGSESKSTSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 520 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKAAASGEGKSSGSGSESKSTGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 4 - protein, 513 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSGSGGSGGSGGSGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTENQVSLTCLVKGFYPSDIAVEWESNGQPENNYKTTPPVLDSDGSFFLYSWLTVDKSRWQEGNVFSCSVMHEALHNHYTQKSLSLSLGKKSRWQEGNVFSCSVMHEALHNHYTQKSLSLSLGK

SEQ ID NO: 5 - protein, 719 aa, synthetic construct
RQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGGGGSGGGGSRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGGGGSGGGGSRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEASESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPRVYTLPPCQEEMTKNQVSLTCLVKGFYPSDIAVEWESNGQPENNYKTTPPVLVSDGSFTLYSRLTVDKSRWQEGNVFSCSVMHEALHNHYTQKSLSLSLGK

SEQ ID NO: 6 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 7 - protein, 191 aa, Homo sapiens
SAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 8 - protein, 152 aa, Homo sapiens
GMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 9 - protein, 156 aa, Homo sapiens
RQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPE

SEQ ID NO: 10 - protein, 347 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 393 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVT

SEQ ID NO: 12 - protein, 347 aa, synthetic construct; identical to SEQ ID NO: 10

SEQ ID NO: 13 - protein, 17 aa, synthetic construct
AAAEGKSSGSGSESKST

SEQ ID NO: 14 - protein, 12 aa, synthetic construct
GSGGSGGSGGSG

SEQ ID NO: 15 - protein, 19 aa, synthetic construct
AAASGEGKSSGSGSESKST

SEQ ID NO: 16 - protein, 10 aa, synthetic construct
GGGGSGGGGS

SEQ ID NO: 17 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 18 - protein, 12 aa, Homo sapiens
ESKYGPPCPPCP

SEQ ID NO: 19 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTENQVSLTCLVKGFYPSDIAVEWESNGQPENNYKTTPPVLDSDGSFFLYSWLTVDKSRWQEGNVFSCSVMHEALHNHYTQKSLSLSLGK

SEQ ID NO: 21 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPRVYTLPPCQEEMTKNQVSLTCLVKGFYPSDIAVEWESNGQPENNYKTTPPVLVSDGSFTLYSRLTVDKSRWQEGNVFSCSVMHEALHNHYTQKSLSLSLGK

SEQ ID NO: 22 - protein, 503 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 23 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 24 - protein, 507 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 25 - protein, 5 aa, synthetic construct
GGGGS

SEQ ID NO: 26 - protein, 6 aa, synthetic construct
GSGGSG

(19) United States
(12) Patent Application Publication
(10) Pub. No.: US 2025/0051441 A1
(43) Pub. Date: Feb. 13, 2025

(54) CD47/4-1BB-TARGETING PROTEIN COMPLEX AND METHODS OF USE THEREOF

(71) Applicant: FBD BIOLOGICS LIMITED, Kowloon (HK)

(72) Inventors: Chun-Yu Lin, Taipei City (TW); Shih-Han Huang, New Taipei City (TW); Yi-Chun HSIEH, Tainan City (TW); Chi-Ling Tseng, Taipei City (TW)

(21) Appl. No.: 18/722,731

(22) PCT Filed: Dec. 9, 2022
(86) PCT No.: PCT/US2022/052395

§ 371 Date: Jun. 21, 2024

Related U.S. Application Data
(60) Provisional application No. 63/293,225, filed on Dec. 23, 2021.

(51) Int. Cl. C07K 16/28

(52) U.S. Cl. CPC C07K 16/2803; C07K 16/2878; C07K 2317/52; C07K 2317/53; C07K 2317/74; C07K 2317/76

(57) ABSTRACT

This disclosure relates to protein complexes targeting CD47 and 4-1BB, and methods of use thereof. In one aspect, the protein complexes include one or more CD47-binding domains including all or a portion of the SIRPα extracellular regions, and one or more 4-1BB-binding domains including all or a portion of the 4-1BBL extracellular region.

### CROSS-REFERENCE TO RELATED APPLICATION

This disclosure claims priority to and benefit of U.S. Provisional Patent Application Ser. No. 63/293,225, filed Dec. 23, 2021, which is incorporated herein by reference in its entirety.

### SEQUENCE LISTING

This application contains a Sequence Listing that has been submitted electronically as an XML file named 52246-0003WO1_SL_ST26.xml. The XML file, created on Dec. 5, 2022, is 30,554 bytes in size. The material in the XML file is hereby incorporated by reference in its entirety.

### TECHNICAL FIELD

This disclosure relates to protein complexes targeting CD47 and 4-1BB, and methods of use thereof.

### BACKGROUND

Signal regulatory protein α (SIRPα) is a regulatory membrane glycoprotein from SIRP family. It is mainly expressed by myeloid cells and also by stem cells or neurons. SIRPα acts as inhibitory receptor and interacts with a broadly expressed transmembrane protein CD47. This interaction negatively controls effector function of innate immune cells such as host cell phagocytosis. SIRPα diffuses laterally on the macrophage membrane and accumulates at a phagocytic synapse to bind CD47, which inhibits the cytoskeleton-intensive process of phagocytosis by the macrophage.

CD47 provides a “do not eat” signal by binding to the N-terminus of signal regulatory protein alpha (SIRPα). CD47 has been found to be overexpressed in many different tumor cells. Targeting CD47 and/or SIRPα can be useful for cancer immunotherapy. However, because CD47 is also expressed on red blood cells (RBCs) and platelets, inhibiting the CD47/SIRPα interaction may cause phagocytosis of RBCs and platelets. Thus, there is a need to develop cancer therapies targeting CD47/SIRPα pathway with limited toxicities.

### SUMMARY

This disclosure relates to protein complexes targeting CD47 and 4-1BB, and methods of use thereof.

In one aspect, the disclosure is related to a protein complex, comprising: (a) an Fc; (b) a CD47-binding domain; and (c) a 4-1BB (tumor necrosis factor receptor superfamily member 9)-binding domain.

In some embodiments, the CD47-binding domain can bind to a cell (e.g., cancer cell) expressing CD47 and/or block the interaction between CD47 and signal regulatory protein α (SIRPα). In some embodiments, the CD47-binding domain is or comprises a SIRPα extracellular domain. In some embodiments, the SIRPα extracellular domain is a human SIRPα extracellular domain. In some embodiments, the CD47-binding domain comprises an amino acid sequence that is at least 80%, 85%, 90%, 95%, 96%, 97%, 98%, 99% or 100% identical to SEQ ID NO: 6.

In some embodiments, the 4-1BB-binding domain can bind to an immune cell (e.g., T cell) expressing 4-1BB and/or stimulate T cell activation and proliferation. In some embodiments, the 4-1BB-binding domain is or comprises a 4-1BBL extracellular domain. In some embodiments, the 4-1BBL extracellular domain is a human 4-1BBL extracellular domain. In some embodiments, the 4-1BB-binding domain comprises an amino acid sequence that is at least 80%, 85%, 90%, 95%, 96%, 97%, 98%, 99% or 100% identical to SEQ ID NO: 7, 8, or 9.

In some embodiments, the Fc is human IgG4 Fc.

In some embodiments, the CD47-binding domain is linked to the N-terminus of a CH2 domain in the Fc, optionally via a hinge region. In some embodiments, the 4-1BB-binding domain is linked to the N-terminus of a CH2 domain in the Fc, optionally via a hinge region. In some embodiments, the hinge region is a human IgG4 hinge region optionally with S228P mutation according to EU numbering. In some embodiments, the 4-1BB-binding domain is linked to the C-terminus of a CH3 domain in the Fc, optionally via a linker peptide. In some embodiments, the Fc comprises a first CH3 domain and a second CH3 domain, in some embodiments, either one or both CH3 domain comprise one or more amino acid residues selected from 360E, 409W, 347R, 399V, and 405T (EU numbering).

In some embodiments, the protein complex comprises two or more CD47-binding domains. In some embodiments, the protein complex comprises two or more 4-1BB-binding domains.

In one aspect, the disclosure is related to a protein complex, comprising (a) a first polypeptide comprising from N-terminus to C-terminus: a first CD47-binding domain, an optional first hinge region, a first Fc region, an optional first linker peptide, a first 4-1BB-binding domain, and a second 4-1BB-binding domain; and (b) a second polypeptide comprising from N-terminus to C-terminus: a second CD47-binding domain, an optional second hinge region, a second Fc region, an optional second linker peptide, and a third 4-1BB-binding domain. In some embodiments, the first CD47-binding domain and/or the second CD47-binding domain comprise a sequence that is at least 80%, 90%, 95%, or 100% identical to SEQ ID NO: 6. In some embodiments, the first 4-1 BB-binding domain, the second 4-1BB binding domain, and/or the third 4-1BB-binding domain comprise a sequence that is at least 80%, 90%, 95%, or 100% identical to SEQ ID NO: 7. In some embodiments, the first hinge region and/or the second hinge region comprise a sequence that is at least 80% identical to SEQ ID NO: 18. In some embodiments, the first Fc region comprises a sequence that is at least 80% identical to SEQ ID NO: 20, and the second Fc region comprises a sequence that is at least 80%, 90%, 95%, or 100% identical to SEQ ID NO: 21. In some embodiments, the first linker peptide and/or the second linker peptide comprise a sequence that is at least 80% identical to SEQ ID NO: 13. In some embodiments, the first 4-1BB-binding domain and the second 4-1BB-binding domain are linked via a third linker peptide, in some embodiments, the third linker peptide comprises a sequence that is at least 80% identical to SEQ ID NO: 14. In some embodiments, the first polypeptide comprises a sequence that is at least 80%, 90%, 95%, or 100% identical to SEQ ID NO: 1, and the second polypeptide comprises a sequence that is at least 80%, 90%, 95%, or 100% identical to SEQ ID NO: 2.

In one aspect, the disclosure is related to a protein complex, comprising (a) a first polypeptide comprising from N-terminus to C-terminus: a first CD47-binding domain, an optional first hinge region, a first Fc region, an optional first linker peptide, and a first 4-1BB-binding domain; and (b) a second polypeptide comprising from N-terminus to C-terminus: a second CD47-binding domain, an optional second hinge region, a second Fc region, an optional second linker peptide, and a second 4-1BB-binding domain. In some embodiments, the first CD47-binding domain and/or the second CD47-binding domain comprise a sequence that is at least 80%, 90%, 95%, or 100% identical to SEQ ID NO: 6. In some embodiments, the first 4-1BB-binding domain and/or the second 4-1BB-binding domain comprise a sequence that is at least 80%, 90%, 95%, or 100% identical to SEQ ID NO: 8. In some embodiments, the first hinge region and/or the second hinge region comprise a sequence that is at least 80% identical to SEQ ID NO: 18. In some embodiments, the first Fc region and the second Fc region comprise a sequence that is at least 80%, 90%, 95%, or 100% identical to SEQ ID NO: 19. In some embodiments, the first linker peptide and/or the second linker peptide comprise a sequence that is at least 80% identical to SEQ ID NO: 15. In some embodiments, the first polypeptide comprises a sequence that is at least 80%, 90%, 95%, or 100% identical to SEQ ID NO: 3, and the second polypeptide comprises a sequence that is at least 80%, 90%, 95%, or 100% identical to SEQ ID NO: 3.

In one aspect, the disclosure is related to a protein complex, comprising (a) a first polypeptide comprising from N-terminus to C-terminus: a first CD47-binding domain, a second CD47-binding domain, an optional first hinge region, and a first Fc region; and (b) a second polypeptide comprising from N-terminus to C-terminus: a first 4-1BB-binding domain, a second 4-1BB-binding domain, a third 4-1BB-binding domain, an optional second hinge region, and a second Fc region. In some embodiments, the first CD47-binding domain and/or the second CD47-binding domain comprise a sequence that is at least 80%, 90%, 95%, or 100% identical to SEQ ID NO: 6. In some embodiments, the first 4-1BB-binding domain, the second 4-1BB binding domain, and/or the third 4-1BB-binding domain comprise a sequence that is at least 80%, 90%, 95%, or 100% identical to SEQ ID NO: 9. In some embodiments, the first hinge region and/or the second hinge region comprise a sequence that is at least 80% identical to SEQ ID NO: 18. In some embodiments, the first Fc region comprises a sequence that is at least 80% identical to SEQ ID NO: 20, and the second Fc region comprises a sequence that is at least 80%, 90%, 95%, or 100% identical to SEQ ID NO: 21. In some embodiments, the first CD47-binding domain and the second 4-1BB-binding domain are linked via a first linker peptide, in some embodiments, the first linker peptide comprises a sequence that is at least 80% identical to SEQ ID NO: 14. In some embodiments, the first 4-1BB-binding domain and the second 4-1BB-binding domain are linked via a second linker peptide, and the second 4-1BB-binding domain and the third 4-1BB-binding domain are linked via a third linker peptide, in some embodiments, the second linker peptide and/or the third linker peptide comprise a sequence that is at least 80% identical to SEQ ID NO: 16. In some embodiments, the first polypeptide comprises a sequence that is at least 80%, 90%, 95%, or 100% identical to SEQ ID NO: 4, and the second polypeptide comprises a sequence that is at least 80%, 90%, 95%, or 100% identical to SEQ ID NO: 5.

In one aspect, the disclosure is related to a nucleic acid comprising a polynucleotide encoding the protein complex as described herein. In some embodiments, the nucleic acid is a DNA (e.g., cDNA) or RNA (e.g., mRNA).

In one aspect, the disclosure is related to a vector comprising one or more of the nucleic acids as described herein.

In one aspect, the disclosure is related to a cell comprising the vector as described herein. In some embodiments, the cell is a CHO cell. In one aspect, the disclosure is related to a cell comprising one or more of the nucleic acids as described herein.

In one aspect, the disclosure is related to a method of producing a protein complex, the method comprising (a) culturing the cell as described herein under conditions sufficient for the cell to produce the protein complex; and (b) collecting the protein complex produced by the cell.

In one aspect, the disclosure is related to an antibody-drug conjugate comprising the protein complex as described herein, covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the protein complex as described herein, or the antibody-drug conjugate as described herein, to the subject. In some embodiments, the subject has a cancer cell expressing CD47. In some embodiments, the cancer is ovarian cancer, head and neck squamous cell cancer, melanoma, non-small cell lung cancer, leukemia, breast cancer, small-cell lung cancer, lymphoma, or colorectal cancer.

In one aspect, the disclosure is related to a method of decreasing the rate of tumor growth, the method comprising contacting a tumor cell with an effective amount of a composition comprising the protein complex as described herein, or the antibody-drug conjugate as described herein.

In one aspect, the disclosure is related to a method of killing a tumor cell, the method comprising contacting a tumor cell with an effective amount of a composition comprising the protein complex as described herein, or the antibody-drug conjugate as described herein.

In one aspect, the disclosure is related to a pharmaceutical composition comprising the protein complex as described herein, and a pharmaceutically acceptable carrier.

As used herein, the term “protein complex” or “protein construct” refers to a complex having one or more polypeptides. In some embodiments, the protein complex has two or more polypeptides, wherein the polypeptides can associate with each other, forming a dimer or a multimer.

As used herein, the term “CD47-binding domain” refers to a protein domain that can bind to CD47. In some embodiments, the CD47-binding domain can be an anti-CD47 antibody, an antigen-binding fragment thereof (e.g., a scFv or a VHH), or a CD47 binding protein or a portion thereof. In some embodiments, the CD47-binding domain can have one or more self-stabilizing domains. In some embodiments, the CD47-binding domain comprises or consists of a SIRPα extracellular domain. The SIRPα can be a wild type SIRPα, a human SIRPα, a polypeptide derived from a wildtype SIRPα (e.g., with mutations), or a portion thereof (e.g., the extracellular region of SIRPα, or IgV domain of SIRPα). In some embodiments, the polypeptide derived from a wildtype SIRPα can have one or more mutations. In some embodiments, the SIRPα extracellular domain comprises or consists of substantially the entire extracellular region of SIRPα or the variant thereof. In some embodiments, the SIRPα extracellular domain comprises or consists of the IgV domain of SIRPα or the variant thereof. In some embodiments, the IgV domain has one or more mutations. In some embodiments, the SIRPα extracellular domain has one or more mutations.

As used herein, the term “4-1BB-binding domain” refers to a protein domain that can bind to 4-1BB. In some embodiments, the 4-1BB-binding domain can be an anti-4-1BB antibody, an antigen-binding fragment thereof (e.g., a scFv or a VHH), or a 4-1BB binding protein or a portion thereof. In some embodiments, the 4-1BB-binding domain can have one or more self-stabilizing domains. In some embodiments, the 4-1BB-binding domain comprises or consists of a 4-1BBL extracellular domain. The 4-1BBL can be a wild type 4-1BBL, a human 4-1BBL, a polypeptide derived from a wildtype 4-1BBL (e.g., with mutations), or a portion thereof (e.g., the extracellular region of 4-1BBL). In some embodiments, the polypeptide derived from a wildtype 4-1BBL can have one or more mutations. In some embodiments, the 4-1BBL extracellular domain comprises or consists of substantially the entire extracellular region of 4-1BBL or the variant thereof. In some embodiments, the 4-1BBL extracellular domain comprises or consists of a portion of the extracellular region of 4-1BBL or the variant thereof. In some embodiments, the 4-1BBL extracellular domain has one or more mutations.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A shows a schematic structure of HCB201-A.

FIG. 1B shows a schematic structure of HCB201-B.

FIG. 1C shows a schematic structure of HCB201-C.

FIG. 2A shows whole cell binding results of HCB201 proteins to CD47 tf CHO-S cells.

FIG. 2B shows whole cell binding results of HCB201 proteins to CD47-expressing FaDu cells.

FIG. 3A shows SIRPα ligand blocking results of HCB201 proteins on CD47 tf CHO-S cells.

FIG. 3B shows SIRPα ligand blocking results of HCB201 proteins on CD47-expressing FaDu cells.

FIG. 4A shows RBC binding results of HCB201 proteins.

FIG. 4B shows platelet binding results of HCB201 proteins.

FIG. 5A shows induced phagocytosis activity of HCB201 proteins by Raw264.7 mouse macrophages against CD47-expressing FaDu cells. HCB201-B and Hu5F9 exhibited the highest phagocytosis activity, followed by SIRPα-G4Fc-wt, HCB201-A, and HCB201-C.

FIG. 5B shows induced phagocytosis activity of HCB201 proteins by Raw264.7 mouse macrophages against human RBC cells. Hu5F9 exhibited a significantly higher phagocytosis activity than HCB201-B, HCB201-C, HCB201-A, and SIRPα-G4Fc-wt.

FIG. 5C shows induced phagocytosis activity of HCB201-B by human monocyte-derived macrophages (MDM) against CD47-expressing Jurkat cells. Hu5F9 exhibited the highest phagocytosis activity, followed by HCB-201-B, and then SIRPα-G4Fc-wt.

FIG. 5D shows induced phagocytosis activity of HCB201-B by human monocyte-derived macrophages (MDM) against human RBC cells. Hu5F9 exhibited a significantly higher phagocytosis activity than HCB201-B and SIRPα-G4Fc-wt.

FIG. 6A shows a schematic diagram of a cell-based reporter assay. The cross-linking effects of HCB201 proteins to induce NF-kB activation in 4-1BB-expressing NF-κB-Luc Jurkat cells were measured.

FIG. 6B shows the bioluminescent signal of 4-1BB-expressing NF-κB-Luc Jurkat cells after cross-linking by HCB201 proteins. The HCB201 proteins were pre-incubated with CD47 tf CHO-S cells.

FIG. 6C shows the bioluminescent signal of 4-1BB-expressing NF-κB-Luc Jurkat cells after cross-linking by HCB201 proteins. The HCB201 proteins were pre-incubated with CD47-expressing FaDu cells.

FIG. 7A shows the concentration of antibodies and proteins used.

FIG. 7B shows the proliferation results after treatment of 3.33 nM, 10 nM, or 30 nM (from left to right) of the tested antibodies or proteins.

FIG. 7C shows the IFN-γ production results of T cells treated by 3.33 nM, 10 nM, or 30 nM (from left to right) of the tested antibodies or proteins. The antibodies (HLX02, PF-05082566, BMS-663513), proteins (SIRPα-G4Fc-wt and peG4Fc-4-1BBL), and fusion protein candidates (HCB201-A and HCB202-B) were added together with 1 μg/ml anti-CD3 antibody (OKT3).

FIG. 7D shows the IL-2 production results of T cells treated by 3.33 nM, 10 nM, or 30 nM (from left to right) of the tested antibodies or proteins. The antibodies (HLX02, PF-05082566, BMS-663513), proteins (SIRPα-G4Fc-wt and peG4Fc-4-1BBL), and fusion protein candidates (HCB201-A and HCB202-B) were added together with 1 μg/ml anti-CD3 antibody (OKT3).

FIGS. 8A-8E show concentrations of released cytokines from PBMCs induced by 6.6 nM or 660 nM immobilized (or plate-bound) HCB201-B. Plate-bound anti-CD3 antibody UCHT-1 and soluble PHA were used as positive controls. Soluble UCHT-1 was used as a negative control. The cytokines included soluble IL-2, IL-6, IL-10, TNFα, and IFN-γ, respectively.

FIGS. 9A-9E show concentrations of released cytokines from PBMCs induced by 6.6 nM or 660 nM soluble HCB201-B. Plate-bound anti-CD3 antibody UCHT-1 and soluble PHA were used as positive controls. Soluble UCHT-1 was used as a negative control. The cytokines included soluble IL-2, IL-6, IL-10, TNF-α, and IFN-γ, respectively.

FIG. 10A shows the secreted TNF-α concentrations by human MDM cells that were treated with 300 nM, 30 nM, or 3 nM (from left to right) HCB201 proteins. Urelumab was used as a positive control. SIRPα-G4Fc-wt and utomilumab were used as negative controls.

FIG. 10B shows the secreted IL-27 concentrations by human MDM cells that were treated with 300 nM, 30 nM, or 3 nM (from left to right) HCB201 proteins. Urelumab was used as a positive control. SIRPα-G4Fc-wt and utomilumab were used as negative controls.

FIGS. 11A-11C show a table summarizing the in vitro assay results.

FIG. 12 lists sequences discussed in the disclosure.

### DETAILED DESCRIPTION

The present disclosure provides protein complexes binding to CD47 and 4-1BB. These protein complexes can be used to target the CD47/SIRPα pathway and 4-1BB/4-1BBL pathway simultaneously. The results indicate that the protein complexes can effectively bind to CD47-expressing cancer cells and block the interaction between endogenous SIRPα and CD47, thereby inducing innate immune response (e.g., phagocytosis of cancer cells by macrophages). On the other hand, the protein complexes showed minimal binding to RBC cells or platelets, thereby inhibiting the clearance of host cells as observed by the anti-CD47 antibody magrolimab. In addition, the protein complexes can stimulate 4-1 BB-expressing immune cell (e.g., T cell) activation, proliferation, and cytokine release. In particular, the protein complexes do not overly induce cytokine release that may lead to hepatotoxicity, which has been observed using the anti-4-1BB antibody urelumab.

Therefore, the protein complexes described herein can be used for cancer treatment with enhanced tumor immunogenicity and antigen presentation through increased phagocytosis by macrophages (e.g., by inactivation of CD47-mediated inhibition of phagocytosis); and enhanced T cell activation through activation of the 4-1BB signaling pathway. Such protein complexes are particularly useful to overcome resistance to anti-PD-1/PD-L1 therapies.

### SIRPα Extracellular Domains

Signal regulatory protein α (SIRPα, SIRPα, Sirpa, or CD172A) is a transmembrane protein. It has an extracellular region comprising three Ig-like domains and a cytoplasmic region containing immunoreceptor tyrosine-based inhibition motifs that mediate binding of the protein tyrosine phosphatases SHP1 and SHP2. Tyrosine phosphorylation of SIRPα is regulated by various growth factors and cytokines as well as by integrin-mediated cell adhesion to extracellular matrix proteins. SIRPα is especially abundant in myeloid cells such as macrophages and dendritic cells, whereas it is expressed at only low levels in T, B, NK, and NKT cells.

The extracellular region of SIRPα can interact with its ligand CD47. The interaction of SIRPα on macrophages with CD47 on red blood cells prevents phagocytosis of Ig-opsonized red blood cells by macrophages in vitro and in vivo. The ligation of SIRPα on phagocytes by CD47 expressed on a neighboring cell results in phosphorylation of SIRPα cytoplasmic immunoreceptor tyrosine-based inhibition motifs, leading to the recruitment of SHP-1 and SHP-2 phosphatases. One resulting downstream effect is the prevention of myosin-IIA accumulation at the phagocytic synapse and consequently inhibition of phagocytosis. Thus, CD47-SIRPα interaction functions as a negative immune checkpoint to send a “don't eat me” signal to ensure that healthy autologous cells are not inappropriately phagocytosed. However, overexpression of CD47 has also been found in nearly all types of tumors, some of which include acute myeloid leukemia, non-Hodgkin's lymphoma, bladder cancer, and breast cancer. Such negative regulation of macrophages can be minimized by blocking the binding of CD47 to SIRPα. Thus, agents blocking CD47/SIRPα interaction can promote both antibody-dependent cellular phagocytosis (ADCP) and in some cases, trigger antibody-dependent cellular cytotoxicity (ADCC), thus can be used to treat various cancers.

Blocking CD47/SIRPα interaction can promote cellular phagocytosis, thus can be used to treat various cancers. It triggers the recognition and elimination of cancer cells by the innate immunity. Agents that target CD47 or SIRPα can be used to treat various tumors and cancers, e.g., solid tumors, hematologic malignancies (e.g., relapsed or refractory hematologic malignancies), acute myeloid leukemia, non-Hodgkin's lymphoma, breast cancer, bladder cancer, ovarian cancer, and small cell lung cancer tumors.

In addition, SIRPα acts to inhibit in vivo clearance of CD47-expressing host cells, including red blood cells and platelets, by macrophages. CD47-SIRPα interactions also appear essential for engraftment upon hematopoietic stem cells. Blocking CD47/SIRPα interaction may cause accidental killing of normal red blood cells, potentially resulting in anemia, and triggering inflammation. Thus, it is important to modulate the interaction of a SIRPα targeting agent with CD47, e.g., with limited or controlled effects on red blood cells.

A detailed description of SIRPα and its function can be found, e.g., in Yanagita et al. “Anti-SIRPα antibodies as a potential new tool for cancer immunotherapy.” JCI insight 2.1 (2017); Seiffert et al. “Signal-regulatory protein α (SIRPα) but not SIRPβ is involved in T-cell activation, binds to CD47 with high affinity, and is expressed on immature CD34+CD38− hematopoietic cells.” Blood 97.9 (2001): 2741-2749; which are incorporated by reference herein in the entirety.

Human SIRPα is a member of signal regulatory proteins (SIRPs). Signal regulatory proteins are cell surface Ig superfamily proteins that mediate essential cell surface protein interactions and signal transduction. SIRPs all contain an N-terminal extracellular region, a single transmembrane domain and a C-terminal intracellular region.

The extracellular region of human SIRPα (UniProt identifier: P78324) has an IgV domain, an Ig-like C1-type 1 domain, and an Ig-like C1-type 2 domain. They correspond to amino acids 32-137, amino acids 148-247, and amino acids 254-348 of the human SIRPα protein (SEQ ID NO: 23; NP_542970.1). Amino acids 1-30 are signal peptides. Human SIRPα also has a long intracellular domain that comprises two putative immunoreceptor tyrosine-based inhibition motifs (ITIM). Activation of SIRPα ITIMs delivers inhibitory signals that negatively regulate cell responses.

In some embodiments, the protein complex comprises one or more CD47-binding domains. In some embodiments, the CD47-binding domain comprises or consists of a SIRPα extracellular domain. As used herein, the “SIRPα extracellular domain” refers to the entire or a portion of the extracellular region of SIRPα, wherein the portion of the extracellular region can bind to CD47. The SIRPα extracellular domain can have one or more protein domains that can fold independently and form self-stabilizing structures. In some embodiments, the SIRPα extracellular domain comprises or consists of one or more domains selected from an IgV domain, an Ig-like C1-type 1 domain, and an Ig-like C1-type 2 domain. In some embodiments, the SIRPα extracellular domain comprises or consists of an IgV domain. In some embodiments, the SIRPα extracellular domain comprises or consists of an IgV domain and an Ig-like C1-type 1 domain. In some embodiments, the SIRPα extracellular domain comprises or consists of an IgV domain, an Ig-like C1-type 1 domain, and an Ig-like C1-type 2 domain.

In some embodiments, the SIRPα extracellular domain described herein includes an amino acid sequence that is at least 80%, 85%, 90%, 95%, 96%, 97%, 98%, 99%, or 100% identical to amino acids 31-148 of human SIRPα protein (GenBank Accession No.: AAH26692.1; SEQ ID NO: 24). In some embodiments, the CD47-binding domain or SIRPα domain described herein includes an amino acid sequence that is at least 80%, 85%, 90%, 95%, 96%, 97%, 98%, 99%, or 100% identical to SEQ ID NO: 6. In some embodiments, the CD47-binding domain or SIRPα extracellular domain described herein includes the IgV domain of human SIRPα protein. In some embodiments, the CD47-binding domain or SIRPα extracellular domain described herein includes the IgV domain of mouse SIRPα protein.

### 4-1BBL Extracellular Domains

4-1BBL (also known as 4-1BB ligand, TNFSF9, CD137L, tumor necrosis factor ligand superfamily member 9) is a type II transmembrane protein of the TNF superfamily primarily on antigen-presenting cells, such as IFN-γ activated macrophages, CD40 ligand activated B cells, monocytes, T cells, dendritic cells (DC), and B cells. 4-1BBL on the cell membrane can transmit a reverse signal, thereby inhibiting the proliferation of activated T cells and inducing their apoptosis. The reverse signal can also induce monocyte activation, promote the secretion of IL-6, IL-8 and TNF-Ade, and prolong cell survival. In addition, the reverse signal can stimulate the maturation of DC derived from CD34+ hematopoietic stem cells. Northern blot analysis revealed multiple 4-1BBL transcripts in brain, placenta, lung, skeletal muscle, and kidney, as well as in activated T cells, transformed B cells, and monocyte lines.

The membranous form of 4-1BBL exists as a trimer, and upon engagement with its receptor on T cells, it delivers a robust costimulatory signal. 4-1BBL was found to be expressed following stimulation on professional APCs including DCs and macrophages as well as activated B cells in both human and mice. Human 4-1BBL message was detected as early as 30 minutes following stimulation through immobilized CD3 monoclonal antibody (mAb) and peaks at 1 hour. 4-1BBL was also present at high levels in the sera of some patients with hematological diseases as well as on some carcinoma cell lines.

A detailed description of 4-1BBL and its function can be found, e.g., in Cheuk, Adam T C, et al., “Role of 4-1BB: 4-1BB ligand in cancer immunotherapy.” Cancer Gene Therapy 11.3 (2004): 215-226; and Li, Yan, et al., “Limited cross-linking of 4-1BB by 4-1BB ligand and the agonist monoclonal antibody Utomilumab.” Cell Reports 25.4 (2018): 909-920; each of which is incorporated by reference in its entirety.

According to the UniProt Database (UniProt ID: P41273), the cytoplasmic region of human TNFSF9 corresponds to amino acids 1-28 of SEQ ID NO: 17, the transmembrane region of human TNFSF9 corresponds to amino acids 29-49 of SEQ ID NO: 17, and the extracellular region of human TNFSF9 corresponds to amino acids 50-254 of SEQ ID NO: 17.

In some embodiments, the protein complex comprises one or more 4-1 BB-binding domains. In some embodiments, the 4-1BB-binding domain comprises or consists of a 4-1BBL extracellular domain. As used herein, the “4-1BBL extracellular domain” refers to the entire or a portion of the extracellular region of 4-1BBL, wherein the portion of the extracellular region can bind to 4-1BB. The 4-1BBL extracellular domain can have one or more protein domains that can fold independently and form self-stabilizing structures.

In some embodiments, the 4-1BB binding domain is a 4-1BBL extracellular domain. In some embodiments, 4-1 BBL extracellular domain described herein includes an amino acid sequence that is at least 80%, 85%, 90%, 95%, 96%, 97%, 98%, 99%, or 100% identical to amino acids 64-254, 90-241, or 88-243 of human 4-1BBL protein (NCBI Accession No.: NP_003802.1; SEQ ID NO: 17). In some embodiments, the 4-1BBL extracellular domain described herein includes an amino acid sequence that is at least 80%, 85%, 90%, 95%, 96%, 97%, 98%, 99%, or 100% identical to SEQ ID NO: 7, 8, or 9. In some embodiments, the 4-1BBL extracellular domain described herein includes a TNF homology domain (THD) that corresponds to amino acids 90-241 or 88-243 of SEQ ID NO: 17.

### Protein Complexes Targeting CD47 and 4-1BB

The disclosure provides protein complexes that can specifically bind to CD47. In some embodiments, these protein complexes can block CD47/SIRPα signaling pathway thus increase immune response. In some embodiments, these protein complexes can initiate phagocytosis.

The disclosure also provides protein complexes that can specifically bind to 4-1BB. In some embodiments, these protein complexes can stimulate 4-1BB/4-1BBL signaling pathway thus increase immune response. In some embodiments, these protein complexes can induce T cell activation, proliferation, and/or cytokine release.

In one aspect, the disclosure provides a protein complex or a protein construct, comprising or consisting of an Fc, one or more CD47-binding domains, and one or more 4-1BB-binding domains. As used herein, the term “Fc” refers to the fragment crystallizable region of an antibody (e.g., IgG, IgE, IgM, IgA, or IgD). The term “Fc region” or “Fc region sequence” refers to heavy chain constant domains (e.g., CH2 and CH3) in a heavy chain peptide that form the Fc region. In some embodiments, the protein complex or the protein construct comprises 1, 2, 3, 4, 5, or 6 CD47-binding domains. In some embodiments, the protein complex or the protein construct comprises 1, 2, 3, 4, 5, or 6 4-1BB-binding domains.

In some embodiments, the protein complex or the protein construct comprises or consists of an Fc, a first domain that specifically binds to cluster of differentiation 47 (CD47), and a second domain that specifically binds to tumor necrosis factor receptor superfamily member 9 (4-1BB).

In some embodiments, the first domain can bind to a cell (e.g., cancer cell) expressing CD47 and/or block the interaction between CD47 and signal regulatory protein α (SIRPα). In some embodiments, the first domain comprises all or a portion of the extracellular region of SIRPα. In some embodiments, the SIRPα is human SIRPα. In some embodiments, the first domain comprises an amino acid sequence that is at least 80%, 85%, 90%, 95%, 96%, 97%, 98%, 99% or 100% identical to SEQ ID NO: 6.

In some embodiments, the second domain can bind to an immune cell (e.g., T cell) expressing 4-1BB and/or stimulate T cell activation and proliferation. In some embodiments, the second domain comprises all or a portion of the extracellular region of tumor necrosis factor ligand superfamily member 9 (4-1BBL). In some embodiments, the 4-1BBL is human 4-1BBL. In some embodiments, the second domain comprises an amino acid sequence that is at least 80%, 85%, 90%, 95%, 96%, 97%, 98%, 99% or 100% identical to SEQ ID NO: 7, 8, or 9.

In some embodiments, the Fc is human IgG4 Fc. In some embodiments, the first domain is linked to the N-terminus of a CH2 domain in the Fc, optionally via a hinge region. In some embodiments, the second domain is linked to the N-terminus of a CH2 domain in the Fc, optionally via a hinge region. In some embodiments, the hinge region is a human IgG4 hinge region optionally with S228P mutation according to EU numbering.

In some embodiments, the second domain is linked to the C-terminus of a CH3 domain in the Fc, optionally via a linker peptide.

In some embodiments, the Fc comprises a first CH3 domain and a second CH3 domain. In some embodiments, each CH3 domain comprises one or more EW-RVT mutations. In some embodiments, the protein complex comprises two or more first domains. In some embodiments, the protein complex comprises two or more second domains.

In some embodiments, the CD47-binding domains or the 4-1BB-binding domains are linked to the Fc region through any of the linker peptide or the hinge region sequence as described herein.

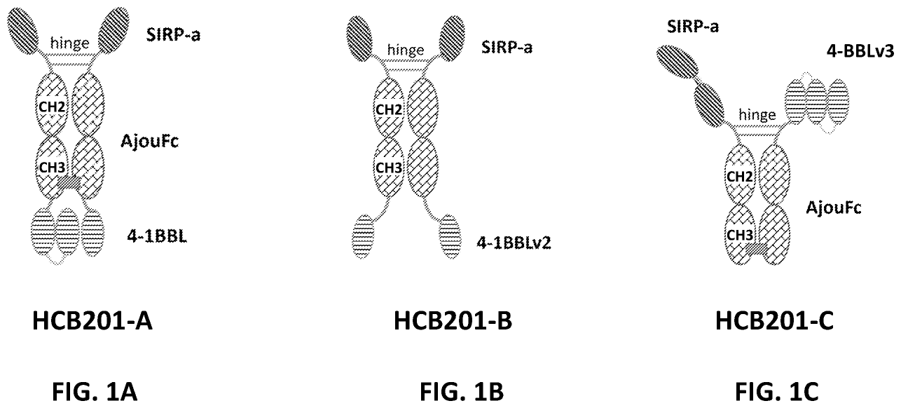

Some embodiments of the protein complexes are shown in FIG. 1. They are described in detail below.

### HCB201-A

In one aspect, the disclosure is related to a protein complex including a first polypeptide and a second polypeptide. The first polypeptide includes, preferably from N-terminus to C-terminus, a first CD47-binding domain, an optional first hinge region, a first Fc region, optionally a first linker peptide, a first 4-1BB-binding domain, and a second 4-1BB-binding domain. The second polypeptide includes, preferably from N-terminus to C-terminus, a second CD47-binding domain, an optional second hinge region, a second Fc region, optionally a second linker peptide, and a third 4-1BB-binding domain. A schematic structure of an exemplary protein complex having a HCB201-A format is shown in FIG. 1A.

In some embodiments, the first and/or the second CD47-binding domains include all or a portion of the extracellular domain of SIRPα, e.g., amino acids 31-148 of human SIRPα protein (GenBank Accession No.: AAH26692. 1; SEQ ID NO: 24); or SEQ ID NO: 6. In some embodiments, the first and the second CD47-binding domains are identical. In some embodiments, the first and/or the second CD47-binding domain include an amino acid sequence that is at least 80%, 85%, 90%, 91%, 92%, 93%, 94%, 95%, 96%, 97%, 98%, 99%, or 100% identical to SEQ ID NO: 6. In some embodiments, the first and/or the second CD47-binding domains include the IgV domain of SIRPα (e.g., human SIRPα). In some embodiments, the SIRPα IgV domain includes one or more mutations. In some embodiments, the first and the second CD47-binding domains are different.

In some embodiments, the first, the second, and/or the third 4-1BB-binding domains include all or a portion of the extracellular domain of 4-1BBL, e.g., amino acids 64-254, 90-241, or 88-243 of human 4-1BBL protein (NCBI Accession No.: NP_003802.1; SEQ ID NO: 17); or any one of SEQ ID NOs: 7-9. In some embodiments, the first, the second, and/or the third 4-1BB-binding domains include a TNF homology domain (THD) that corresponds to amino acids 90-241 or 88-243 of SEQ ID NO: 17. In some embodiments, the first, the second, and/or the third 4-1BB-binding domains are identical. In some embodiments, the first, the second, and/or the third 4-1 BB-binding domains are different. In some embodiments, the first, the second, and/or the third 4-1BB-binding domains include an amino acid sequence that is at least 80%, 85%, 90%, 91%, 92%, 93%, 94%, 95%, 96%, 97%, 98%, 99%, or 100% identical to SEQ ID NO: 7, 8, or 9. In some embodiments, the first 4-1BB-binding domain and the second 4-1BB-binding domain are linked via a third linker peptide. In some embodiments, the first, second, and/or the third 4-1BB-binding domains can form an intramolecular trimer.

In some embodiments, the first and/or the second hinge region include all or a portion of the hinge region of an immunoglobulin, e.g., human IgG4 hinge region (SEQ ID NO: 18). In some embodiments, the first and/or the second hinge region include an amino acid sequence that is at least 80%, 85%, 90%, 91%, 92%, 93%, 94%, 95%, 96%, 97%, 98%, 99%, or 100% identical to SEQ ID NO: 18. In some embodiments, the first and the second hinge regions are identical. In some embodiments, the first and the second hinge regions are different. In some embodiments, the first and/or the second hinge region include a proline at position 228 according to EU numbering.

In some embodiments, the first and/or the second Fc region are identical and can form a Fc homodimer. In some embodiments, the first and/or the second Fc region include all or a portion of the Fc region of an immunoglobulin, e.g., human IgG4 Fc region (SEQ ID NO: 19). In some embodiments, the first and/or the second Fc region are different. In some embodiments, the first and/or the second Fc region can form a Fc heterodimer by introducing one or more mutations. In some cases, the first and/or the second Fc region can include one or more EW/RVT mutations. For example, the first Fc region can include a glutamate at position 360 and a tryptophan at position 409 according to EU numbering; the second Fc region can include an arginine at position 347, a valine at position 399, and a threonine at position 405 according to EU numbering. In some embodiments, the second Fc region also include a cysteine at position 454 according to EU numbering for formation of a disulfide bond with the first Fc region. In some embodiments, the first Fc region includes an amino acid sequence that is at least 80%, 85%, 90%, 91%, 92%, 93%, 94%, 95%, 96%, 97%, 98%, 99%, or 100% identical to SEQ ID NO: 20, and the second Fc region includes an amino acid sequence that is at least 80%, 85%, 90%, 91%, 92%, 93%, 94%, 95%, 96%, 97%, 98%, 99%, or 100% identical to SEQ ID NO: 21. In some embodiments, the first and/or the second Fc region can form a Fc heterodimer using other technologies, e.g., by introducing one or more knob-into-hole (KIH) residues. Details of the EW-RVT and other heterodimeric Fc technologies can be found, e.g., in Ha, et al. “Immunoglobulin Fc heterodimer platform technology: from design to applications in therapeutic antibodies and proteins.” Frontiers In Immunology 7 (2016): 394, which is incorporated herein by reference in its entirety.

In some embodiments, the first, the second, and/or the third linker peptide described herein include an amino acid sequence that is at least 80%, 85%, 90%, 95%, or 100% identical to any one of SEQ ID NOs: 13-16. In some embodiments, the first, the second, and/or the third linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 25) or GSGGSG (SEQ ID NO: 26).

In some embodiments, at least 1, 2, 3, 4, or 5 amino acid residues (e.g., an alanine and a serine, from N-terminus to C-terminus) are inserted between the first CD47-binding domain and the first hinge region. In some embodiments, at least 1, 2, 3, 4, or 5 amino acid residues (e.g., an alanine and a serine, from N-terminus to C-terminus) are inserted between the second CD47-binding domain and the second hinge region. In some embodiments, the at least 1, 2, 3, 4, or 5 amino acid residues are encoded by a sequence for restriction enzyme digestion (e.g., NheI).

In some embodiments, the first polypeptide includes an amino acid sequence that is at least 80%, 85%, 90%, 91%, 92%, 93%, 94%, 95%, 96%, 97%, 98%, 99%, or 100% identical to SEQ ID NO: 1. In some embodiments, the second polypeptide includes an amino acid sequence that is at least 80%, 85%, 90%, 91%, 92%, 93%, 94%, 95%, 96%, 97%, 98%, 99%, or 100% identical to SEQ ID NO: 2.

### HCB201-B

In one aspect, the disclosure is related to a protein complex including a first polypeptide and a second polypeptide. The first polypeptide includes, preferably from N-terminus to C-terminus, a first CD47-binding domain, an optional first hinge region, a first Fc region, optionally a first linker peptide, and a first 4-1BB-binding domain. The second polypeptide includes, preferably from N-terminus to C-terminus, a second CD47-binding domain, an optional second hinge region, a second Fc region, optionally a second linker peptide, and a second 4-1BB-binding domain. A schematic structure of an exemplary protein complex having a HCB201-B format is shown in FIG. 1B.

In some embodiments, the first and/or the second CD47-binding domains include all or a portion of the extracellular domain of SIRPα, e.g., amino acids 31-148 of human SIRPα protein (GenBank Accession No.: AAH26692.1; SEQ ID NO: 24); or SEQ ID NO: 6. In some embodiments, the first and the second CD47-binding domains are identical. In some embodiments, the first and/or the second CD47-binding domains include an amino acid sequence that is at least 80%, 85%, 90%, 91%, 92%, 93%, 94%, 95%, 96%, 97%, 98%, 99%, or 100% identical to SEQ ID NO: 6. In some embodiments, the first and/or the second CD47-binding domains include the IgV domain of SIRPα (e.g., human SIRPα). In some embodiments, the SIRPα IgV domain includes one or more mutations. In some embodiments, the first and the second CD47-binding domains are different.

In some embodiments, the first and/or the second 4-1BB-binding domains include all or a portion of the extracellular domain of 4-1BBL, e.g., amino acids 64-254, 90-241, or 88-243 of human 4-1BBL protein (NCBI Accession No.: NP_003802.1; SEQ ID NO: 17); or any one of SEQ ID NOs: 7-9. In some embodiments, the first and/or the second 4-1BB-binding domains include a TNF homology domain (THD) that corresponds to amino acids 90-241 or 88-243 of SEQ ID NO: 17. In some embodiments, the first and/or the second 4-1BB-binding domains are identical. In some embodiments, the first and/or the second 4-1 BB-binding domains are different. In some embodiments, the first and/or the second 4-1BB-binding domains include an amino acid sequence that is at least 80%, 85%, 90%, 91%, 92%, 93%, 94%, 95%, 96%, 97%, 98%, 99%, or 100% identical to SEQ ID NO: 7, 8, or 9. In some embodiments, three protein complexes described herein can form an intermolecular trimer of the first 4-1BB-binding domains. In some embodiments, three protein complexes described herein can form an intermolecular trimer of the second 4-1BB-binding domains.

In some embodiments, the first and/or the second hinge region include all or a portion of the hinge region of an immunoglobulin, e.g., human IgG4 hinge region (SEQ ID NO: 18). In some embodiments, the first and/or the second hinge region include an amino acid sequence that is at least 80%, 85%, 90%, 91%, 92%, 93%, 94%, 95%, 96%, 97%, 98%, 99%, or 100% identical to SEQ ID NO: 18. In some embodiments, the first and the second hinge regions are identical. In some embodiments, the first and the second hinge regions are different. In some embodiments, the first and/or the second hinge region include a proline at position 228 according to EU numbering.

In some embodiments, the first and/or the second Fc region are identical and can form a Fc homodimer. In some embodiments, the first and/or the second Fc region include all or a portion of the Fc region of an immunoglobulin, e.g., human IgG4 Fc region (SEQ ID NO: 19). In some embodiments, the first and/or the second Fc region are different. In some embodiments, the first and/or the second Fc region can form a Fc heterodimer by introducing one or more mutations. In some cases, the first and/or the second Fc region can include one or more EW/RVT mutations. For example, the first Fc region can include a glutamate at position 360 and a tryptophan at position 409 according to EU numbering; the second Fc region can include an arginine at position 347, a valine at position 399, and a threonine at position 405 according to EU numbering. In some embodiments, the second Fc region also include a cysteine at position 454 according to EU numbering for formation of a disulfide bond with the first Fc region. In some embodiments, the first Fc region includes an amino acid sequence that is at least 80%, 85%, 90%, 91%, 92%, 93%, 94%, 95%, 96%, 97%, 98%, 99%, or 100% identical to SEQ ID NO: 20, and the second Fc region includes an amino acid sequence that is at least 80%, 85%, 90%, 91%, 92%, 93%, 94%, 95%, 96%, 97%, 98%, 99%, or 100% identical to SEQ ID NO: 21. In some embodiments, the first and/or the second Fc region can form a Fc heterodimer using other technologies, e.g., by introducing one or more knob-into-hole (KIH) residues.

In some embodiments, the first and/or the second linker peptide described herein include an amino acid sequence that is at least 80%, 85%, 90%, 95%, or 100% identical to any one of SEQ ID NOs: 13-16. In some embodiments, the first and/or the second linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 25) or GSGGSG (SEQ ID NO: 26).

In some embodiments, at least 1, 2, 3, 4, or 5 amino acid residues (e.g., an alanine and a serine, from N-terminus to C-terminus) are inserted between the first CD47-binding domain and the first hinge region. In some embodiments, at least 1, 2, 3, 4, or 5 amino acid residues (e.g., an alanine and a serine, from N-terminus to C-terminus) are inserted between the second CD47-binding domain and the second hinge region. In some embodiments, the at least 1, 2, 3, 4, or 5 amino acid residues are encoded by a sequence for restriction enzyme digestion (e.g., NheI).

In some embodiments, the first polypeptide includes an amino acid sequence that is at least 80%, 85%, 90%, 91%, 92%, 93%, 94%, 95%, 96%, 97%, 98%, 99%, or 100% identical to SEQ ID NO: 3. In some embodiments, the second polypeptide includes an amino acid sequence that is at least 80%, 85%, 90%, 91%, 92%, 93%, 94%, 95%, 96%, 97%, 98%, 99%, or 100% identical to SEQ ID NO: 3.

### HCB201-C

In one aspect, the disclosure is related to a protein complex including a first polypeptide and a second polypeptide. The first polypeptide includes, preferably from N-terminus to C-terminus, a first CD47-binding domain, a second CD47-binding domain, an optional first hinge region, and a first Fc region. The second polypeptide includes, preferably from N-terminus to C-terminus, a first 4-1BB-binding domain, a second 4-1BB-binding domain, a third 4-1BB-binding domain, an optional second hinge region, and a second Fc region. A schematic structure of an exemplary protein complex having a HCB201-C format is shown in FIG. 1C.

In some embodiments, the first and/or the second CD47-binding domains include all or a portion of the extracellular domain of SIRPα, e.g., amino acids 31-148 of human SIRPα protein (GenBank Accession No.: AAH26692.1; SEQ ID NO: 24); or SEQ ID NO: 6. In some embodiments, the first and the second CD47-binding domains are identical. In some embodiments, the first and/or the second CD47-binding domains include an amino acid sequence that is at least 80%, 85%, 90%, 91%, 92%, 93%, 94%, 95%, 96%, 97%, 98%, 99%, or 100% identical to SEQ ID NO: 6. In some embodiments, the first and/or the second CD47-binding domains include the IgV domain of SIRPα (e.g., human SIRPα). In some embodiments, the SIRPα IgV domain includes one or more mutations. In some embodiments, the first and the second CD47-binding domains are different. In some embodiments, the first and the second CD47-binding domains are linked via a first linker peptide.

In some embodiments, the first, the second, and/or the third 4-1BB-binding domains include all or a portion of the extracellular domain of 4-1BBL, e.g., amino acids 64-254, 90-241, or 88-243 of human 4-1BBL protein (NCBI Accession No.: NP_003802.1; SEQ ID NO: 17); or any one of SEQ ID NOs: 7-9. In some embodiments, the first, the second, and/or the third 4-1BB-binding domains include a TNF homology domain (THD) that corresponds to amino acids 90-241 or 88-243 of SEQ ID NO: 17. In some embodiments, the first, the second, and/or the third 4-1BB-binding domains are identical. In some embodiments, the first, the second, and/or the third 4-1BB-binding domains are different. In some embodiments, the first, the second, and/or the third 4-1BB-binding domains include an amino acid sequence that is at least 80%, 85%, 90%, 91%, 92%, 93%, 94%, 95%, 96%, 97%, 98%, 99%, or 100% identical to SEQ ID NO: 7, 8, or 9. In some embodiments, the first, the second, and/or the third 4-1BB-binding domains can form a intramolecular trimer. In some embodiments, the first and the second 4-1BB-binding domains are linked via a second linker peptide. In some embodiments, the second and the third 4-1BB-binding domains are linked via a third linker peptide.

In some embodiments, the first and/or the second hinge region include all or a portion of the hinge region of an immunoglobulin, e.g., human IgG4 hinge region (SEQ ID NO: 18). In some embodiments, the first and/or the second hinge region include an amino acid sequence that is at least 80%, 85%, 90%, 91%, 92%, 93%, 94%, 95%, 96%, 97%, 98%, 99%, or 100% identical to SEQ ID NO: 18. In some embodiments, the first and the second hinge regions are identical. In some embodiments, the first and the second hinge regions are different. In some embodiments, the first and/or the second hinge region include a proline at position 228 according to EU numbering.

In some embodiments, the first and/or the second Fc region are identical and can form a Fc homodimer. In some embodiments, the first and/or second Fc region include all or a portion of the Fc region of an immunoglobulin, e.g., human IgG4 Fc region (SEQ ID NO: 19). In some embodiments, the first and/or the second Fc region are different. In some embodiments, the first and/or the second Fc region can form a Fc heterodimer by introducing one or more mutations. In some cases, the first and/or the second Fc region can include one or more EW/RVT mutations. For example, the first Fc region can include a glutamate at position 360 and a tryptophan at position 409 according to EU numbering; the second Fc region can include an arginine at position 347, a valine at position 399, and a threonine at position 405 according to EU numbering. In some embodiments, the second Fc region also include a cysteine at position 454 according to EU numbering for formation of a disulfide bond with the first Fc region. In some embodiments, the first Fc region includes an amino acid sequence that is at least 80%, 85%, 90%, 91%, 92%, 93%, 94%, 95%, 96%, 97%, 98%, 99%, or 100% identical to SEQ ID NO: 20, and the second Fc region includes an amino acid sequence that is at least 80%, 85%, 90%, 91%, 92%, 93%, 94%, 95%, 96%, 97%, 98%, 99%, or 100% identical to SEQ ID NO: 21. In some embodiments, the first and/or second Fc region can form a Fc heterodimer using other technologies, e.g., by introducing one or more knob-into-hole (KIH) residues.

In some embodiments, the first, the second, and/or the third linker peptide described herein include an amino acid sequence that is at least 80%, 85%, 90%, 95%, or 100% identical to any one of SEQ ID NOs: 13-16. In some embodiments, the first, second, and/or third linker peptide described herein include an amino acid sequence that is at least 80%, 85%, 90%, 95%, or 100% identical to one or more (e.g., 1, 2, 3, 4, 5, 6, 7, or 8) repeats of GGGGS (SEQ ID NO: 25) or GSGGSG (SEQ ID NO: 26).

In some embodiments, at least 1, 2, 3, 4, or 5 amino acid residues (e.g., an alanine and a serine, from N-terminus to C-terminus) are inserted between the second CD47-binding domain and the first hinge region. In some embodiments, at least 1, 2, 3, 4, or 5 amino acid residues (e.g., an alanine and a serine, from N-terminus to C-terminus) are inserted between the third 4-1BB-binding domain and the second hinge region. In some embodiments, the at least 1, 2, 3, 4, or 5 amino acid residues are encoded by a sequence for restriction enzyme digestion (e.g., NheI).

In some embodiments, the first polypeptide includes an amino acid sequence that is at least 80%, 85%, 90%, 91%, 92%, 93%, 94%, 95%, 96%, 97%, 98%, 99%, or 100% identical to SEQ ID NO: 4. In some embodiments, the second polypeptide includes an amino acid sequence that is at least 80%, 85%, 90%, 91%, 92%, 93%, 94%, 95%, 96%, 97%, 98%, 99%, or 100% identical to SEQ ID NO: 5.

### Characteristics of Protein Complexes

In some embodiments, the protein complex can comprise any CD47-binding domains and/or 4-1BB-binding domains as described herein. The disclosure also provides nucleic acid comprising a polynucleotide encoding a polypeptide described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The protein complex described herein can include an Fc of an antibody. These antibodies can be of any type (e.g., IgG, IgE, IgM, IgD, IgA, and IgY), class or subclass (e.g., IgG1, IgG2, IgG3, IgG4, IgA1, IgA2, IgE1, IgE2). In some embodiments, the Fc region is derived from human IgG (e.g., IgG1, IgG2, IgG3, or IgG4). In some embodiments, the Fc region is an IgG4 Fc region (e.g., human IgG4 Fc region).

In some embodiments, the protein complex described herein is linked to the Fc region through an antibody hinge region (e.g., IgG, IgE hinge region). In addition, the Fc region can be modified to provide desired effector functions or serum half-life.

The protein complex described herein can block the binding between CD47 and endogenous SIRPα that are expressed on immune cells. In some embodiments, by binding to CD47, the protein complex described herein can inhibit the binding of CD47 (e.g., that is expressed on tumor cells) to endogenous SIRPα that is expressed on immune cells (e.g., myeloid cells, macrophages and dendritic cells), thereby blocking CD47/SIRPα pathway, upregulating immune response, and promoting phagocytosis.

In some embodiments, the protein complex described herein can increase immune response, activity or number of immune cells (e.g., myeloid cells, macrophages, dendritic cells, antigen presenting cells) by at least 10%, 20%, 30%, 40%, 50%, 60%, 70%, 80%, 90%, 100%, 2 folds, 3 folds, 5 folds, 10 folds, or 20 folds.

In some implementations, the protein complex described herein can bind to CD47 (e.g., human CD47, monkey CD47, or mouse CD47) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms. Affinities can be deduced from the quotient of the kinetic rate constants (KD-koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, or 10 pM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

In some implementations, the protein complex described herein can bind to 4-1BB (e.g., human 4-1BB, monkey 4-1BB, or mouse 4-1BB) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms. Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, or 10 pM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity include, e.g., ELISA, RIA, and surface plasmon resonance (SPR). In some embodiments, the protein complex described herein can bind to monkey CD47, and/or mouse CD47. In some embodiments, the protein complex described herein cannot bind to monkey CD47, and/or mouse CD47.

In some embodiments, thermal stabilities are determined. The protein complex described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the protein complex described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the protein complex described herein has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI(%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the tumor inhibitory effects of the protein complex described herein are comparable to an anti-CD47 reference antibody, e.g., Hu5F9-G4, or an anti-SIRPα antibody, e.g., CC-95251. Hu5F9-G4 is described e.g., in Sikic et al. “First-in-human, first-in-class phase I trial of the anti-CD47 antibody Hu5F9-G4 in patients with advanced cancers.” Journal of Clinical Oncology 37.12 (2019): 946, which is incorporated herein by reference in its entirety. In some embodiments, the tumor inhibitory effects of the protein complex described herein are at least 10%, 20%, 30%, 40%, 50%, 60%, 70%, 80%, 90%, 1 fold, 2 folds, or 5 folds more than an anti-CD47 reference antibody, e.g., Hu5F9-G4, or an anti-SIRPα antibody, e.g., CC-95251. In some embodiments, the tumor inhibitory effects of the protein complex described herein are at least 10%, 20%, 30%, 40%, 50%, 60%, 70%, 80%, 90%, 1 fold, 2 folds, or 5 folds more than hSIRPα-G4Fc-wt (Trillium). Details of hSIRPα-G4Fc-wt (Trillium), also named TTI-622. Amino acid sequence of hSIRPα-G4Fc-wt (Trillium) is shown in SEQ ID NO: 11.

In some embodiments, the protein complex described herein has a functional Fc. In some embodiments, the Fc is from human IgG1, human IgG2, human IgG3, or human IgG4. In some embodiments, effector function of a functional Fc is antibody-dependent cell-mediated cytotoxicity (ADCC). In some embodiments, effector function of a functional Fc is phagocytosis. In some embodiments, effector function of a functional Fc is ADCC and phagocytosis. In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the main peak of HPLC-SEC accounts for at least 80%, 85%, 90%, 95%, 96%, 97%, 98%, 99%, or 99.5% of the protein complex described herein after purification by protein A-based affinity chromatography and/or size-exclusive chromatography.

In some embodiments, the protein complex described herein can bind to human CD47-expressing tumor cells (e.g., human CD47 tf CHO-S cells, or FaDu cells) with an affinity that is at least 30%, at least 40%, at least 50%, at least 60%, at least 70%, at least 80%, at least 90%, at least 100%, at least 110%, or at least 120% as compared to that an anti-CD47 reference antibody (e.g., Hu5F9).

In some embodiments, the protein complex described herein can bind to RBC cells or platelets (e.g., from human donors) with an affinity that is less than 90%, less than 80%, less than 70%, less than 60%, less than 50%, less than 40%, less than 30%, less than 20%, less than 10%, less than 5%, less than 3%, or less than 1% as compared to that of an anti-CD47 reference antibody (e.g., Hu5F9).

In some embodiments, the protein complex described herein does not induce hemagglutination. In some embodiments, the protein complex described herein can induce hemagglutination at a minimal concentration that is greater than 500-fold, 2000-fold, 5000-fold, 20000-fold, or 50000-fold as compared to that of an anti-CD47 reference antibody (e.g., Hu5F9).

In some embodiments, the protein complex described herein can block the interaction between CD47 (e.g., human CD47 or fragments thereof) and SIRPα (e.g., human SIRPα or fragments thereof). In some embodiments, the protein complex described herein can block the interaction between human CD47-expressing cells (e.g., CD47 tf CHO-S cells or FaDu cells) and human SIRPα. In some embodiments, the blocking ability of the protein complex described herein is at least at least 50%, at least 60%, at least 70%, at least 80%, at least 90%, at least 100%, at least 110%, at least 120%, at least 130%, at least 140%, or at least 150% as compared to that an anti-CD47 reference antibody (e.g., Hu5F9).

In some embodiments, the protein complex described herein can induce phagocytosis of CD47-expressing tumor cells (e.g., FaDu cells) by mouse macrophages (e.g., Raw264.7 cells). In some embodiments, EC50 values of the protein complex described herein to induce phagocytosis of CD47-expressing tumor cells is less than 30 nM, less than 20 nM, less than nM, less than 5 nM, less than 4 nM, less than 3 nM, less than 2 nM, or less than 1 nM. In some embodiments, EC50) values of the protein complex described herein to induce phagocytosis of CD47-expressing tumor cells is comparable (e.g., at least 80%, 85%, 90%, or 95%) to that of an anti-CD47 reference antibody (e.g., Hu5F9). In some embodiments, the protein complex described herein has a weaker ability (e.g., less than 80%, 70%, 60%, 50%, 40%, 30%, 20% or 10%) to induce phagocytosis of RBC cells by mouse macrophages (e.g., Raw 264.7 cells) than an anti-CD47 reference antibody (e.g., Hu5F9).

In some embodiments, the protein complex described herein can induce phagocytosis of CD47-expressing tumor cells (e.g., Jurkat cells) by human macrophages (e.g., MDM cells). In some embodiments, the ability to induce phagocytosis of the protein complex described herein is comparable (at least 60%, 65%, 70%, 75%, 80%, 85%, 90%, or 95%) to that of an anti-CD47 reference antibody (e.g., Hu5F9). In some embodiments, the protein complex described herein has a weaker ability (less than 80%, 70%, 60%, 50%, 40%, 30%, 20% or 10%) to induce phagocytosis of RBC cells by human macrophages (e.g., MDM cells) than an anti-CD47 reference antibody (e.g., Hu5F9).

Endogenous expression of CD47 on a variety of cell types, including red blood cells, creates a formidable “antigen sink” that may limit the efficacy of CD47-targeting therapies. Thus, the weaker ability of the protein complex described herein to induce phagocytosis of RBC cells and/or platelets may increase the in vivo efficacy of the protein complex. In addition, the protein complex may be administered with a lower dose level and/or less frequent dosage schedule than an anti-CD47 reference antibody (e.g., Hu5F9).

In some embodiments, the protein complex described herein can bring immune cells (e.g., T cells expressing 4-1BB on the cell surface) and cancer cells (e.g., CD47-expressing cancer cells) into close proximity, thereby facilitating elimination of cancer cells and promote immune cell activation, proliferation, and/or cytokine release.

In some embodiments, the protein complex described herein (e.g., HCB201-B) can activate 4-1BB-expressing immune cells (e.g., T cells) in the presence of CD47-expressing cancer cells. In some embodiments, the immune cell activation ability of the protein complex described herein is at least 1-fold, 2-fold, 5-fold, 10-fold, 20-fold, 50-fold, 100-fold, 500-fold, or 1000-fold higher than that of hSIRPα-G4Fc-wt (Trillium).

In some embodiments, the protein complex described herein can promote T cell proliferation that are pre-activated, e.g., by CD3/CD28 beads. For example, the protein complex can increase the pre-activated T cell number by at least 10%, 20%, 30%, 40%, 50%, 60%, 70%, 80%, 90%, or 100% than control antibodies (e.g., anti-4-1BB antibodies utomilumab and urelumab; or hSIRPα-G4Fc-wt (Trillium TTI-662)). In some embodiments, the protein complex described herein can increase cytokine (e.g., IFN-γ and/or IL-2) production by at least 1-fold, 2-fold, 5-fold, 10-fold, 20-fold, 50-fold, 100-fold, 200-fold, 500-fold, 1000-fold, 2000-fold, or 10000-fold than control antibodies (e.g., anti-4-1BB antibodies utomilumab and urelumab; or hSIRPα-G4Fc-wt (Trillium TTI-662)).

In some embodiments, cytokines (e.g., IL-2, IL-6, IL-10, TNF-α, and/or IFN-γ) released from human PBMCs induced by the protein complex described herein are less than 30%, 20%, 10%, 9%, 8%, 7%, 6%, 5%, 4%, 3%, 2%, or 1% as compared to that induced by immobilized anti-CD3 (UCHT-1) or soluble form of PHA. In some embodiments, cytokines (e.g., TNF-α and IL-27) secreted from macrophages (e.g., MDM cells) induced by the protein complex described herein are less than 80%, 70%, 60%, 50%, 40%, 30%, 20%, or 10% as compared to that induced by the anti-4-1BB antibody urelumab.

In some embodiments, the protein complex described herein does not induce cytokine storm in human. In some embodiments, the protein complex described herein is not a superagonist. Details of cytokine storm and superagonist can be found, e.g., in Shimabukuro-Vornhagen, A. et al. “Cytokine release syndrome.” Journal for ImmunoTherapy of Cancer 6.1 (2018): 1-14, which is incorporated herein by reference in its entirety.

In some embodiments, the protein complex described herein can inhibit tumor growth.

In some embodiments, the linker peptides described herein can increase the solubility of the HCB201 proteins as compared to the same molecule without the linker peptides. In some embodiments, the linker peptides described herein can increase the flexibility of the linked domains (e.g., any of the 4-1BB-binding domains or CD-47-binding domains) as compared to the same molecule without the linker peptides.

### Methods of Making Protein Complexes

Variants of the protein complexes described herein can be prepared by introducing appropriate nucleotide changes into the DNA encoding a polypeptide or a part thereof or by peptide synthesis. Such variants include, for example, deletions, insertions, or substitutions of residues within the amino acids sequences.

Screening can be performed to increase binding affinity of the CD47-binding domains and 4-1BB binding domains. Any combination of deletions, insertions, and/or combinations can be made to arrive at a variant that has increased binding affinity for the target. The amino acid changes introduced into the variant can also alter or introduce new post-translational modifications into the polypeptide, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

The CD47-binding domains and/or 4-1BB binding domains can be derived from any species of animal, including mammals. Non-limiting examples of binding domain variants include sequences derived from humans, primates, e.g., monkeys and apes, cows, pigs, horses, sheep, camelids (e.g., camels and llamas), chicken, goats, and rodents (e.g., rats, mice, hamsters and rabbits).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. Techniques for incorporating DNA into such expression systems are well known to those of ordinary skill in the art. The DNA may also be “naked.” The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding a polypeptide of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270), the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptides can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The protein constructs or polypeptides of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow; retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of protein constructs or polypeptides disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the protein constructs or the polypeptides, vector comprising the polynucleotide encoding the protein constructs or the polypeptides, and/or compositions thereof is to be administered, a severity of symptoms and a route of administration, and thus administration can be determined on an individual basis.

An effective amount can be administered in one or more administrations. By way of example, an effective amount of the protein constructs or the polypeptides is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the protein constructs or the polypeptides used.

Effective amounts and schedules for administering the protein constructs or the polypeptides, the polynucleotides encoding the protein constructs or the polypeptides, and/or compositions disclosed herein may be determined empirically, and making such determinations is within the skill in the art. Those skilled in the art will understand that the dosage that must be administered will vary depending on, for example, the mammal that will receive the protein constructs or the polypeptides, the polynucleotides, and/or compositions disclosed herein, the route of administration, the particular type of polynucleotides, and/or compositions disclosed herein used and other drugs being administered to the mammal.

A typical daily dosage of an effective amount of the protein constructs and/or the polypeptides is 0.1 mg/kg to 100 mg/kg (mg per kg of patient weight). In some embodiments, the dosage can be less than 100 mg/kg. 10 mg/kg, 9 mg/kg, 8 mg/kg, 7 mg/kg. 6 mg/kg. 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg. 8 mg/kg. 7 mg/kg, 6 mg/kg, 5 mg/kg, 4 mg/kg. 3 mg/kg, 2 mg/kg, 1 mg/kg, 0.5 mg/kg, or 0.1 mg/kg. In some embodiments, the dosage is about 10 mg/kg. 9 mg/kg. 8 mg/kg. 7 mg/kg, 6 mg/kg, 5 mg/kg, 4 mg/kg, 3 mg/kg, 2 mg/kg, or 1 mg/kg. In some embodiments, the dosage is about 1 to 10 mg/kg, about 1 to 5 mg/kg, or about 2 to 5 mg/kg.

In any of the methods described herein, the protein constructs or the polypeptides can be administered to the subject at least once a week (e.g., once a week, twice a week, three times a week, four times a week, once a day, twice a day; or three times a day).

In some embodiments, the one or more additional therapeutic agents can be administered to the subject prior to, or after administering the protein constructs or the polypeptides. In some embodiments, the one or more additional therapeutic agents are administered to the subject such that there is an overlap in the bioactive period of the one or more additional therapeutic agents and the protein constructs or the polypeptides in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10), a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-SIRPα antibody, an anti-CD47 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CD20 antibody (e.g., rituximab) or an anti-EGF receptor antibody (e.g., cetuximab).

### Pharmaceutical Compositions and Routes of Administration

Also provided herein are pharmaceutical compositions that contain the protein constructs or the polypeptides described herein. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the agents can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing the protein constructs or the polypeptides described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the agents can be formulated in aqueous solutions, preferably in physiologically-compatible buffers to reduce discomfort at the site of injection. The solution can contain formulatory agents such as suspending, stabilizing and/or dispersing agents. Alternatively the protein constructs or the polypeptides can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50) (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the protein constructs or the polypeptides described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents can vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life in vivo.

The pharmaceutical compositions can be included in a container, pack, or dispenser together with instructions for administration. The disclosure also provides methods of manufacturing the protein constructs or the polypeptides for various uses as described herein.

### EXAMPLES

The invention is further described in the following examples, which do not limit the scope of the invention described in the claims.

### Example 1. Design of Fc-Based Designer Biologics (FBDB™) with SIRPα×4-1BBL Formats

Different dual-targeting formats of FBDB™ were designed and developed to (1) improve the antigen presentation by macrophages, and (2) enhance the re-activation of exhausted T cells within tumors.

Each format contains at least two different types of binding domains that are directly or indirectly connected to the Fc region of an IgG (e.g., human IgG4). The two types of binding domains are: (1) one or more SIRPα extracellular domains that can stimulate the antigen-presentation by inducing phagocytosis; (2) one or more 4-1BBL extracellular domains that can bind to 4-1BB on tumor-antigen specific T cells, stimulating their expansion, cytokine production, and the development of cytolytic effector functions. For example, the SIRPα extracellular domains can reverse the inhibition of macrophages by blocking the interaction between CD47 on tumor cells and SIRPα on macrophages; the 4-1BBL extracellular domains can bind to it receptor 4-1BB on exhausted T cells and provide a co-stimulatory signal for T cell activation and expansion.

Three dual-targeting formats of FBDB™ were designed as shown in FIGS. 1A-1C. HCB201-A (schematic structure shown in FIG. 1A) includes a first polypeptide chain and a second polypeptide chain with amino acid sequences set forth in SEQ ID NO: 1 and SEQ ID NO: 2, respectively. Both polypeptide chains include a human IgG4 hinge region, a CH2 region and a CH3 region of human IgG4 Fc, whereas the CH3 region of the first polypeptide chain includes K360E and R409W mutations according to EU numbering, and the CH3 region of the second polypeptide chain includes Q347R, D399V, and F405T mutations according to EU numbering. The first and second polypeptide chains are also named EW and RVT chains, respectively. The two chains can form an EW-RVT Fc heterodimer (or “AjouFc”). In addition, the first polypeptide chain includes a SIRPα extracellular domain (SEQ ID NO: 6; with sequence identical to amino acids 31-148 of human SIRPα protein (GenBank Accession No.: AAH26692.1; SEQ ID NO: 24)) that is connected to the N-terminus of the hinge region of human IgG4 (with S228P mutation according to EU numbering), and two 4-1BBL extracellular domains (SEQ ID NO: 7: with sequence identical to amino acids 64-254 of human 4-1BBL protein (NCBI Accession No.: NP_003802.1; SEQ ID NO: 17)) that are connected to the C-terminus of the CH3 region of the human IgG4 Fc via a linker peptide (SEQ ID NO: 13). The two 4-1BBL extracellular domains are connected via a linker peptide (SEQ ID NO: 14). The second polypeptide chain includes a SIRPα extracellular domain (SEQ ID NO: 6) that is connected to the N-terminus of the hinge region of the human IgG4 (with S228P mutation according to EU numbering), and one 4-1BBL extracellular domain (SEQ ID NO: 7) that is connected to the C-terminus of the CH3 region of the human IgG4 Fc via a linker peptide (SEQ ID NO: 13).

HCB201-B (schematic structure shown in FIG. 1B) includes two identical polypeptide chains, and each polypeptide chain has an amino acid sequence set forth in SEQ ID NO: 3. Specifically, each polypeptide chain includes a human IgG4 hinge region, a CH2 region, and a CH3 region of human IgG4 Fc. A SIRPα extracellular domain (SEQ ID NO: 6) is connected to the N-terminus of the hinge region of human IgG4 (with S228P mutation according to EU numbering), and one 4-1BBLv2 extracellular domain (SEQ ID NO: 8: with sequence identical to amino acids 90-241 of human 4-1BBL protein (NCBI Accession No.: NP_003802.1; SEQ ID NO: 17)) is connected to the C-terminus of the CH3 domain of human IgG4 Fc via a linker peptide (SEQ ID NO: 15).

HCB201-C (schematic structure shown in FIG. 1C) includes a first polypeptide chain and a second polypeptide chain with amino acid sequences set forth in SEQ ID NO: 4 and SEQ ID NO: 5, respectively. Both polypeptide chains include a human IgG4 hinge region, a CH2 region, and a CH3 region of human IgG4 Fc, whereas the CH3 region of the first polypeptide chain includes K360E and K409W mutations according to EU numbering, and the CH3 region of the second polypeptide chain includes Q347R. D399V. and F405T mutations according to EU numbering. Thus, the first and second polypeptide chains are also named EW and RVT chains, respectively. The two chains can form an EW-RVT Fc heterodimer (or “AjouFc”). In addition, the first polypeptide chain includes two SIRPα extracellular domains (SEQ ID NO: 6) that are connected to the N-terminus of the hinge region of human IgG4 (with S228P mutation according to EU numbering). The two SIRPα extracellular domains are connected via a linker peptide (SEQ ID NO: 14). The second polypeptide includes three 4-1BBLv3 extracellular domains (SEQ ID NO: 9; with sequence identical to amino acids 88-243 of human 4-1BBL protein (NCBI Accession No.: NP_003802.1; SEQ ID NO: 17)). The three 4-1BBLv3 extracellular domains are connected via two linker peptides (SEQ ID NO: 16).

The expressed proteins were purified by a protein A column, followed by HPLC-SEC (high-performance liquid chromatography coupled with size-exclusion chromatography; Agilent), and the percentage of high molecular weight peaks (HMW %), the percentage of the main peak (Main %), and the percentage of low molecular weight peaks (LMW %) were measured. As shown in the table below, the HCB201 proteins can be harvested with high purity. In addition, the amino acid sequences of HCB201-A, HCB201-B, and HCB201-C were analyzed using the deimmunization tool (Immune Epitope Database And Analysis Resource; Dhanda et al. “Development of a strategy and computational application to select candidate protein analogues with reduced HLA binding and immunogenicity.” Immunology 153.1 (2018): 118-132) to identify immunogenic regions. No immunogenicity was identified.

TABLE 1

| | MW | | After SEC Fractionation | | |
|---|---|---|---|---|---|
| Format | (kDa) | pI | HMW % | Major % | LMW % |
| HCB201-A | 141.565 | 6.24 | 13.9 | 84.47 | 1.63 |
| HCB201-B | 112.839 | 6.71 | 2.90 | 97.10 | 0 |
| HCB201-C | 120.090 | 6.90 | 6.28 | 93.72 | 0 |

### Example 2. Determination of the Whole Cell Binding Ability to CD47 tf CHO-S and CD47-Expressing Tumor Cells

To determine the whole cell binding ability of HCB201 proteins to CD47 expressed on cell surface, transfected CHO-S cells expressing human CD47 (CD47 tf CHO-S) or hypopharyngeal carcinoma FaDu cells were used as target cells. The cells were incubated with serially diluted HCB201 proteins (at indicated concentrations) in FACS buffer (phosphate-buffered saline (PBS) supplemented with 4% fetal bovine serum (FBS)) at 4° C. for 30 minutes. After the incubation, the cells were washed twice with FACS buffer. Afterwards, the cells were incubated with R-Phycoerythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA, USA).

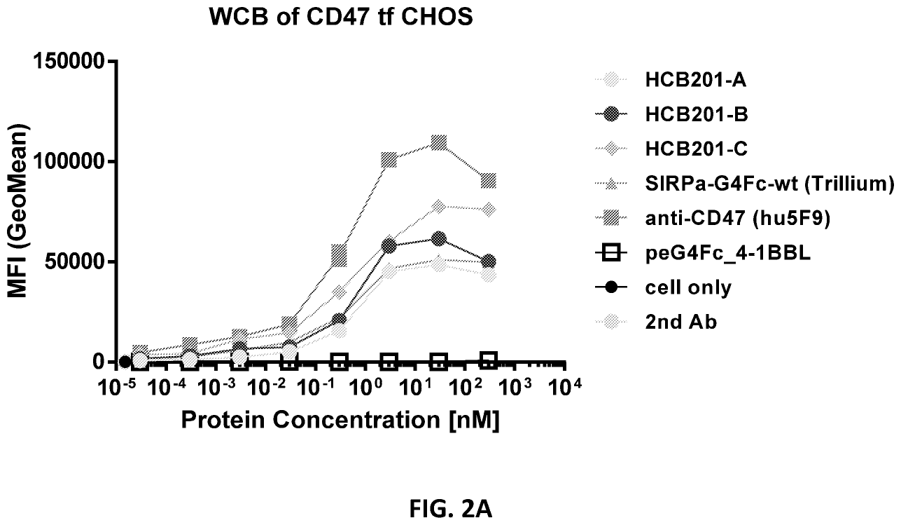

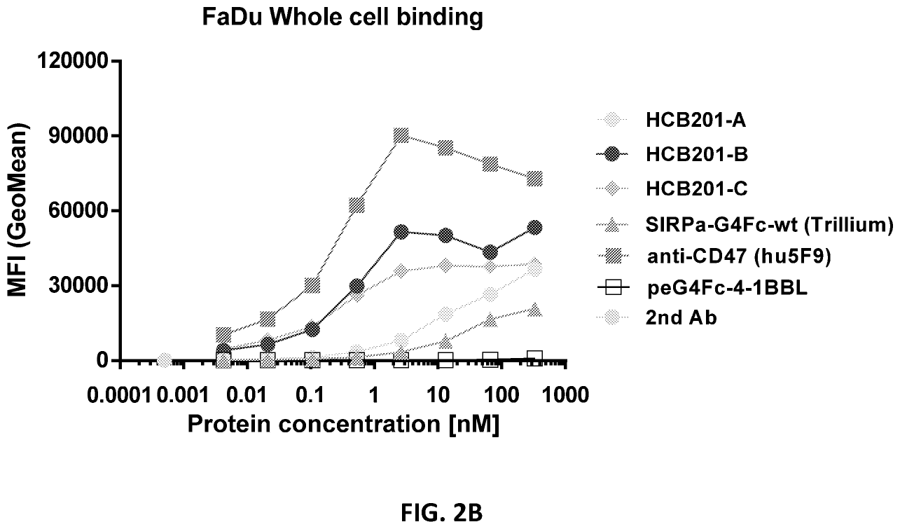

As shown in FIGS. 2A-2B. HCB201-B and HCB201-C exhibited a higher binding ability to CD47-expressing cells than HCB201-A. An anti-CD47 reference antibody Hu5F9 and SIRPα-G4Fc-wt (Trillium TTI-662) were used as positive controls. SIRPα-G4Fc-wt includes two identical polypeptide chains, and each having an amino acid sequence shown in SEQ ID NO: 10. Specifically, each polypeptide chain includes, from N-terminus to C-terminus, a SIRPα extracellular region, a human IgG4 hinge region, and a human IgG4 Fc, peG4Fc-4-1BBL was used as a negative control, peG4Fc-4-1BBL includes two identical polypeptide chains, and each having an amino acid sequence shown in SEQ ID NO: 11. Specifically, each polypeptide chain includes, from N-terminus to C-terminus, a human IgG4 hinge region, a human IgG4 Fc, and a 4-1BBL extracellular region. “pe” stands for the expression vector pEE12.4.

### Example 3. Determination of the SIRPα Ligand Blocking Ability

To determine the SIRPα ligand blocking ability of HCB201 proteins, flow cytometry-based assays were performed using CD47 tf CHO-S or FaDu cells as the target cells. Specifically, the HCB201 proteins were serially diluted at indicated concentrations in FACS buffer (PBS supplemented with 4% FBS). Biotinylated SIRPα-Fc (amino acid sequence shown in SEQ ID NO: 12) was added to FACS buffer to final concentrations of 10-20 nM. Equal volume of the HCB201 protein solution and biotinylated SIRPα-Fc solution were mixed and added to the target cells at 4° C. for 30 minutes. The cells were washed with FACS buffer twice, followed by a 30-minute incubation with streptavidin PE (eBioscience, CA, USA). The samples were analyzed using a CytoFLEX flow cytometer (Beckman Coulter Inc., CA, USA). Hu5F9) and SIRPα-G4Fc-wt (Trillium TTI-662) were used as positive controls. peG4Fc-4-1BBL was used as a negative control.

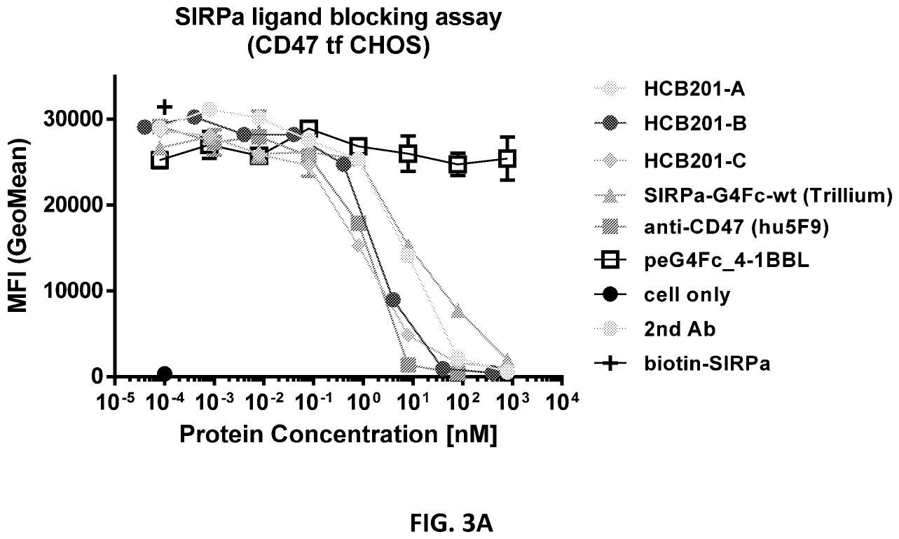

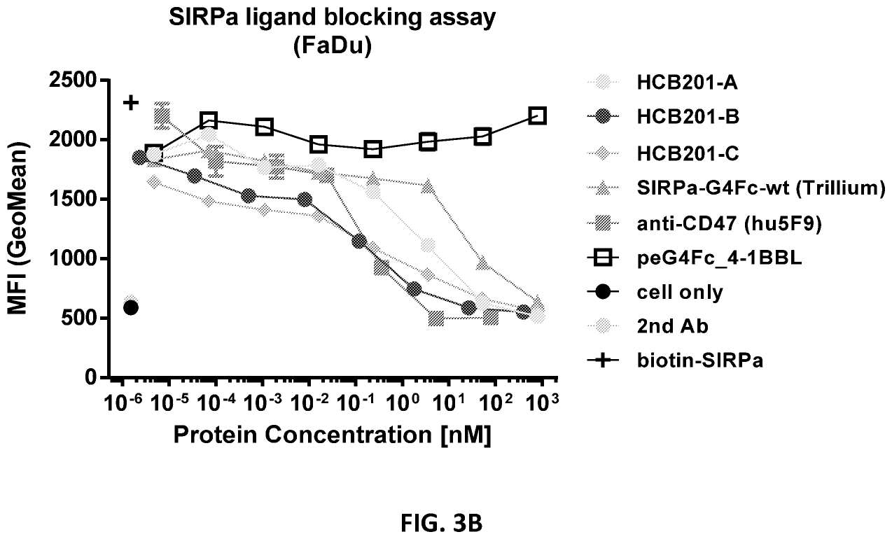

As shown in FIGS. 3A-3B, HCB201-B and HCB201-C showed similar SIRPα ligand blocking activities as compared to the anti-CD47 reference antibody Hu5F9. In addition, the SIRPα ligand blocking activity of HCB201-B and HCB201-C were more potent than that of HCB201-A.

### Example 4. Determination of the Whole Cell Binding Ability to RBCs or Platelets

To determine the RBC or platelet binding ability of HCB201 proteins, human RBCs or platelets were incubated with serially diluted HCB201 proteins at indicated concentrations in FACS buffer (PBS supplemented with 4% FBS) at 4° C. for 30 minutes. After the incubation, the RBCs or platelets were washed twice with FACS buffer. Afterwards, the cells were incubated with R-Phycoerythrin-AffiniPure Goat Anti-Human IgG (Jackson ImmunoResearch, Cat #: 109-115-098) at 4° C. for 30 minutes. The samples were analyzed using a CytoFLEX flow cytometer (Beckman Coulter Inc., CA. USA). Hu5F9 and SIRPα-G4Fc-wt (Trillium TTI-662) were used as positive controls. peG4Fc-4-1BBL was used as a negative control.

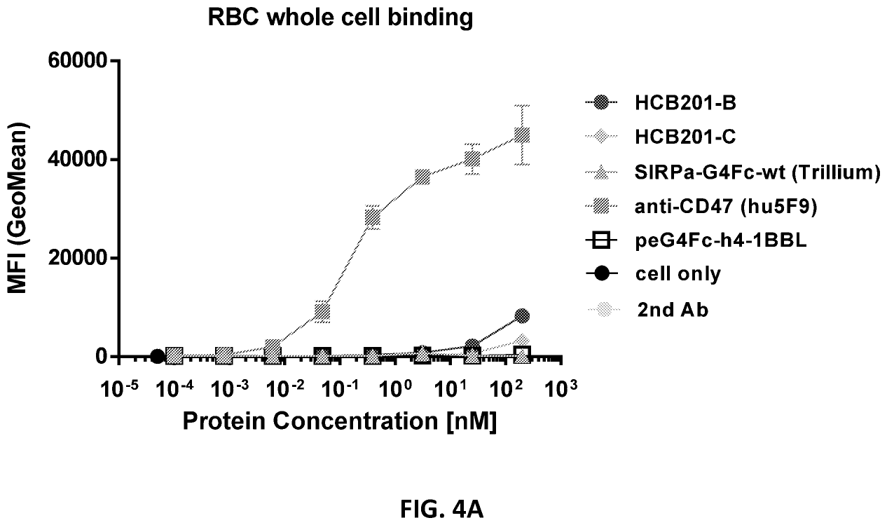

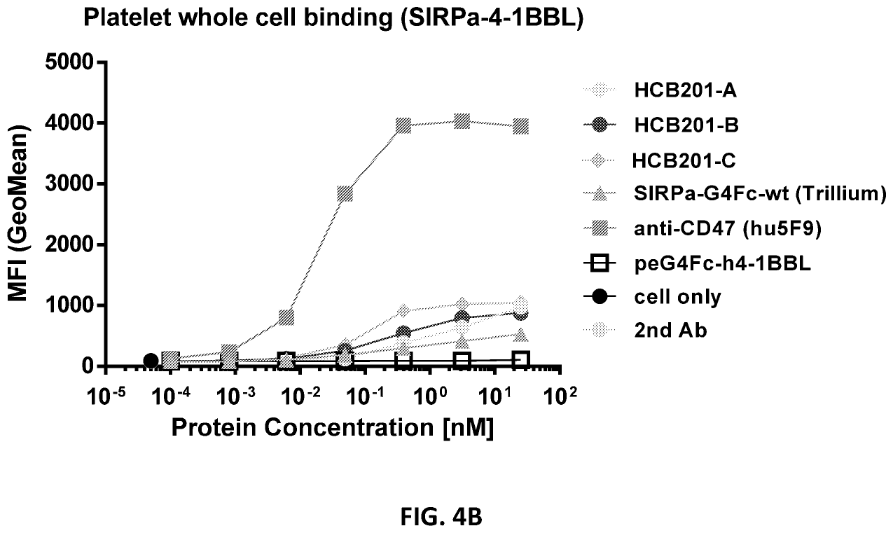

As shown in FIGS. 4A-4B. HCB201-A. HCB201-B. and HCB201-C exhibited a substantially weaker RBC and platelet binding activity than the anti-CD47 reference antibody Hu5F9, indicating that the HCB201 proteins have a lower risk to induce receptor-mediated endocytosis, also known as “antigen sink.” which is a major side effects induced by Hu5F9. Compared to other lymphocytes. RBCs and platelets constitute a majority of cell population in the blood. High RBC or platelet binding can affect the pharmacokinetics and localization to the tumor site. Here, the low binding activity to RBCs and platelets of the HCB201 proteins can reduce this side effect and enhance the chance of tumor targeting. Furthermore, the low binding activity of the HCB201 proteins to RBCs can also decrease the phagocytosis of RBCs by macrophages, thereby decreasing the major side effect as observed using Hu5F9.

### Example 5. Induction of Phagocytosis of CD47-Expressing Cancer Cells or RBCs by Macrophages

To determine the macrophage-mediated phagocytosis induced by HCB201 proteins on cancer cells or RBCs, phagocytosis assay were performed as follows. Briefly, the CD47-expressing Jurkat or FaDu cells were labeled with CFSE (carboxyfluorescein succinimidyl ester) dye and pre-incubated with the HCB201 proteins at indicated concentrations at 37° C. for 30 minutes. The solutions were then incubated with Raw 264.7 mouse microphages or human monocyte-derived macrophages (MDM) at a 1:1 cell-to-cell ratio at 37° C. for 2 hours. After the incubation, the mouse macrophages were stained with PE-Cyanine 7-conjugated F4/80 antibody (eBioscience) and the human macrophages were stained with PE-Cyanine 7-conjugated CD14 antibody (eBioscience), respectively. The ability of the HCB201 proteins to induce phagocytosis was evaluated by calculating the percentage of CFSE+F4/80+ cells in total F4/80+ cells (for Raw264.7) or the percentage of CFSE+CD14+ cells in total CD14+ cells (for MDM).

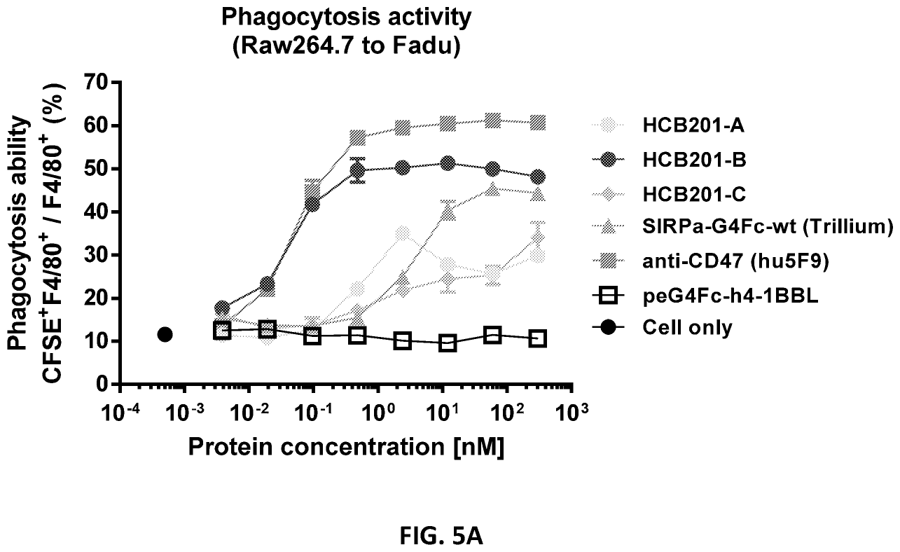

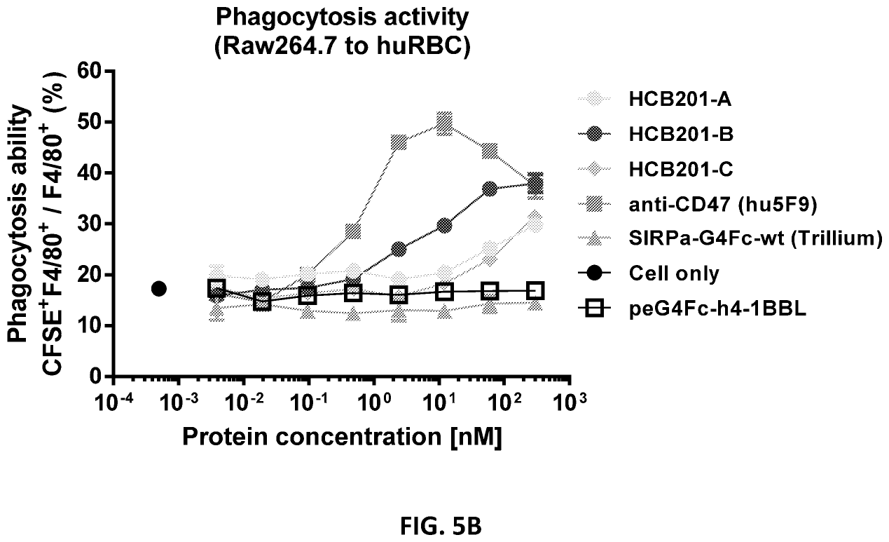

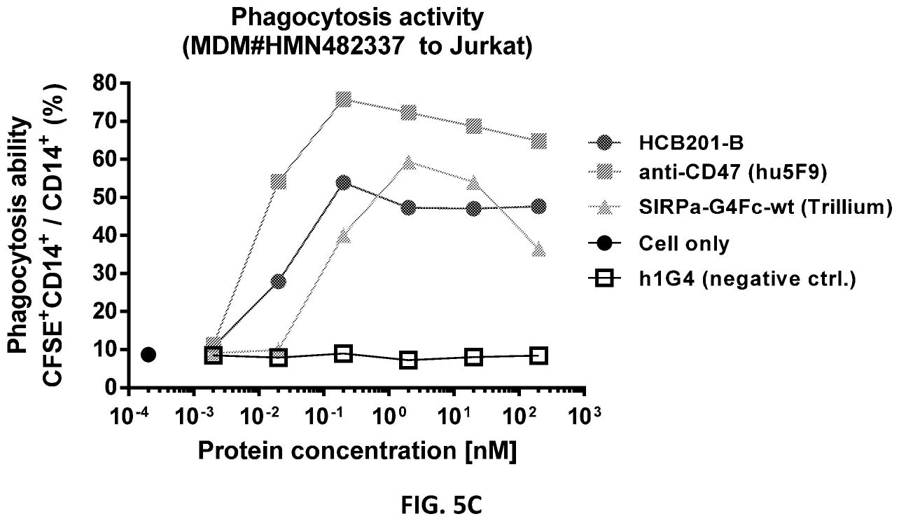

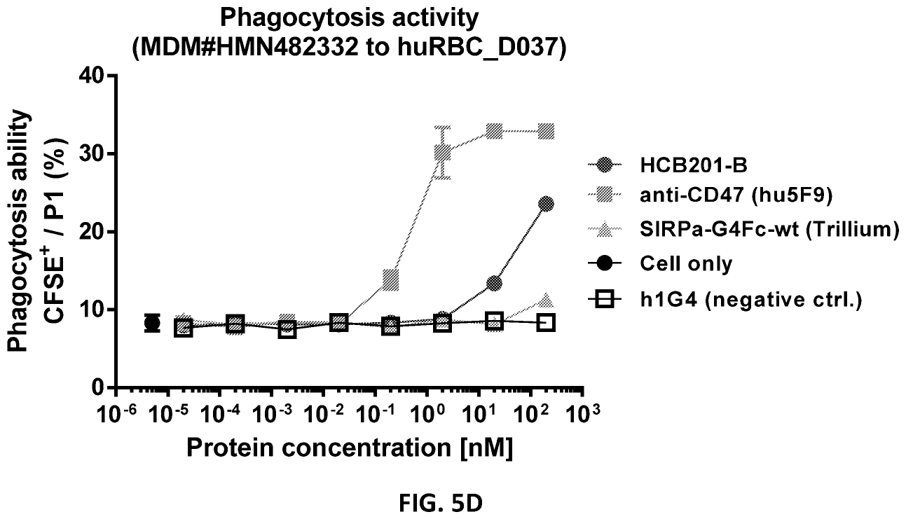

As shown in FIGS. 5A-5B. HCB201-B exhibited similar phagocytosis activity on CD47-expressing FaDu cells and much weaker phagocytosis activity on RBCs as compared to the anti-CD47 reference antibody Hu5F9. As shown in FIGS. 5C-5D. HCB201-B exhibited comparable MDM-mediated phagocytosis activity on CD47-expressing Jurkat cells and relatively weaker phagocytosis activity on RBCs as compared to Hu5F9. These results indicate that HCB201-B can induce substantial phagocytosis on cancer cells and retain a better safety profile than the anti-CD47 antibody Hu5F9.

### Example 6. Verification of HCB201-B-Triggered Reporter Activity on 4-1BB-Transfected Cells

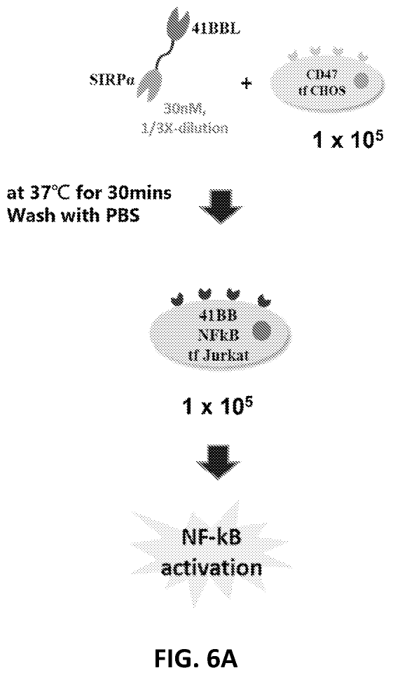

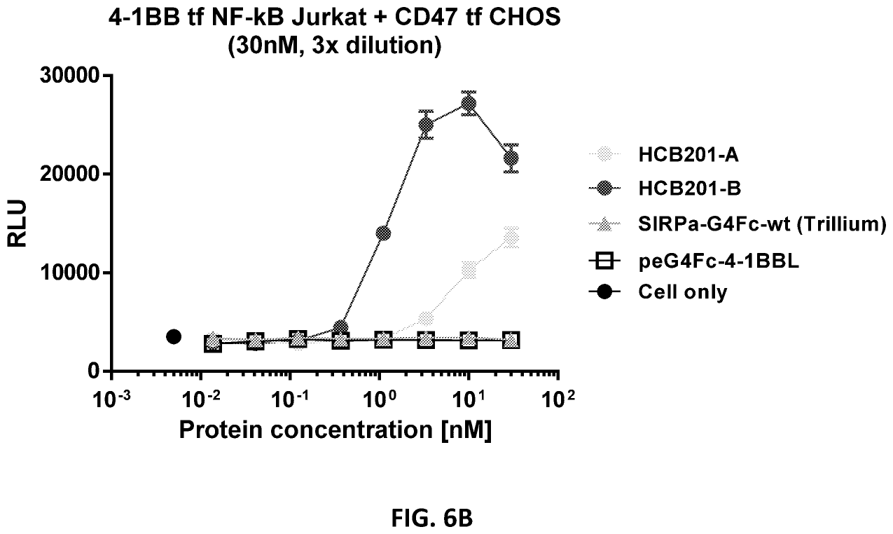

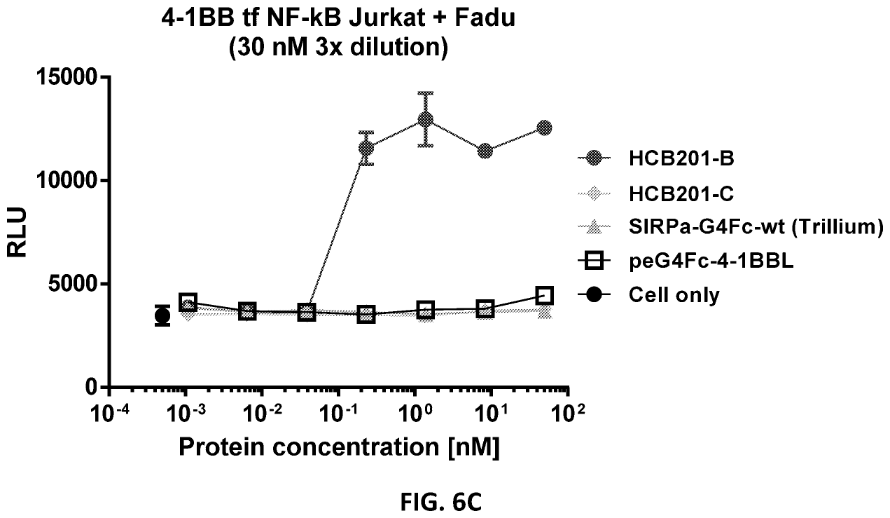

A cell-based reporter assay was designed to determine the NF-kB activity induced by HCB201-B through cross-linking effects. FIG. 6A shows a schematic diagram of the experimental procedures. Specifically, 30 nM of HCB201-B was serially diluted (3-fold) at indicated concentrations and incubated at 37° C. for 30 minutes with 1×10⁵ CD47-expressing CHO-S or FaDu cells. After the incubation, the CD47 tf CHOS or FaDu cells were washed with PBS, and then incubated with 1×10⁵ of 4-1BB-expressing NF-κB-Luc Jurkat cells for 5 hours. Cross-linking between the CD47-expressing CHO-S or FaDu cells and 4-1BB-expressing NF-κB-Luc Jurkat by HCB201-B can induce significant activation of NF-κB, which can be detected using a bioluminescent detector.

As shown in FIG. 6B, HCB201-B significantly triggered the NF-κB reporter activity through the SIRPα-CD47-mediated cross-linking activity on CD47-expressing CHO-S cells and 4-1BB-expressing NF-κB-Luc Jurkat cells. As shown in FIG. 6C, HCB201-B significantly triggered the NF-κB reporter activity through the SIRPα-CD47-mediated cross-linking activity on CD47-expressing FaDu cells and 4-1 BB-expressing NF-κB-Luc Jurkat cells. SIRPα-G4Fc-wt (Trillium TTI-662) and peG4Fc-4-1BBL were used as negative controls. The results indicate that HCB201-B can greatly trigger the reporter activity of 4-1BB-expressing NF-kB-Luc Jurkat cells through SIRPα-CD47-mediated cross-linking activity.

### Example 7. Induction of T Cell Proliferation and Cytokine Release

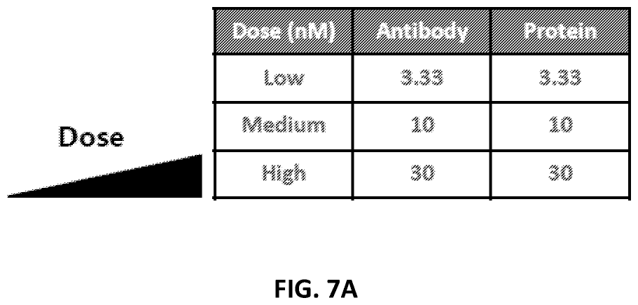

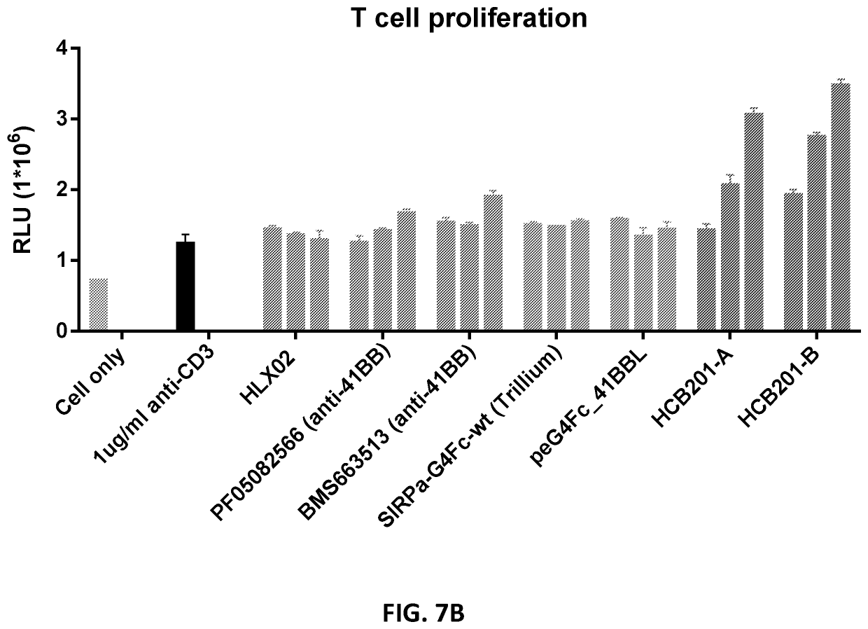

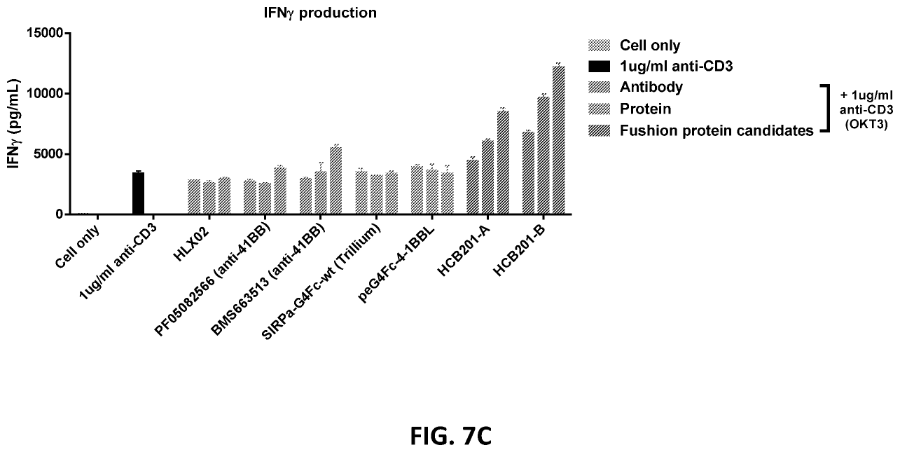

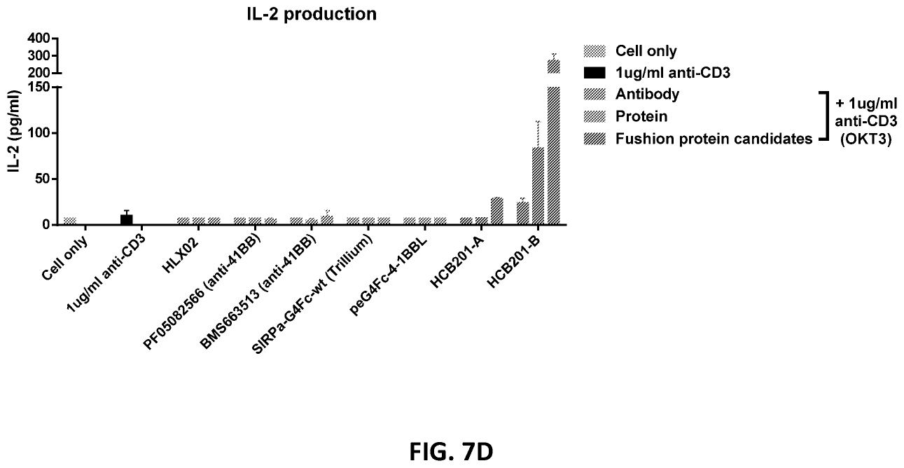

T cell proliferation and cytokine release induced by HCB201 proteins were determined as follows. As shown in FIG. 7A, the antibodies or proteins were first diluted to 3.33 nM (low dose), 10 nM (medium dose), or 30 nM (high dose). Briefly, 1×10⁵/well of T cells were pre-activated by CD3/CD28 beads at a 1:1 cell-to-bead ratio for 3 days. Pre-activated T cells were then added into a 96-well plate and incubated with the antibodies or proteins at indicated concentrations for another 5 days. After the incubation, the cells were collected for determination of T cell proliferation by the CellTiter-Glo® cell viability assay and the culture supernatants were collected for IL-2 and IFN-γ determination. The amount of IL-2 and IFN-γ was measured by Human IL-2 ELISA MAXIM Deluxe and Human IFN-γ ELISA MAX™ Deluxe kits (BioLegend) according to the manufacturer's instructions. SIRPα-G4Fc-wt (Trillium TTI-662), peG4Fc-4-1BBL, and HLX02 (an anti-HER2 antibody developing by Henlius) were used as negative controls. Anti-4-1BB antibodies utomilumab (PF-05082566) and urelumab (BMS-663513) were used as reference antibodies.

As shown in FIG. 7B, HCB201-A and HCB201-B significantly induced T cell proliferation as compared to the negative controls. As shown in FIG. 7C, HCB201-A and HCB201-B significantly increased INF-γ production as compared to the INF-γ production level of T cells treated with the anti-CD3 antibody alone or together with other antibodies or proteins. As shown in FIG. 7D, HCB201-B significantly induced IL-2 production as compared to the IL-2 production level of T cells treated with the anti-CD3 antibody alone or together with other antibodies or proteins. The results indicate that HCB201-B can greatly induce T cell proliferation and cytokine release.

### Example 8. Induction of Cytokine Release from Human PBMCs

To evaluate the potential of HCB201-B to induce cytokine release syndrome (CRS) in vitro, cytokine release assays (CRAs) were performed as follows. Briefly, the human PBMCs (peripheral blood mononuclear cells) were incubated with HCB201-B that were immobilized or soluble in a 96-well plate at a concentration of 660 nM or 6.6 nM for 3 days. Concentrations of soluble IL-2, IL-6, IL-10, TNF-α, and IFN-γ released from PBMCs were measured by the Multi-Analyte Flow Assay (LEGENDplex™, BioLegend) according to the manufacturer's instructions. Soluble form of PHA (Polyhydroxyalkanoates) and immobilized anti-CD3 (UCHT-1) were used as positive controls. The immuno-stimulation activity of UCHT-1 can be triggered upon cross-linking.

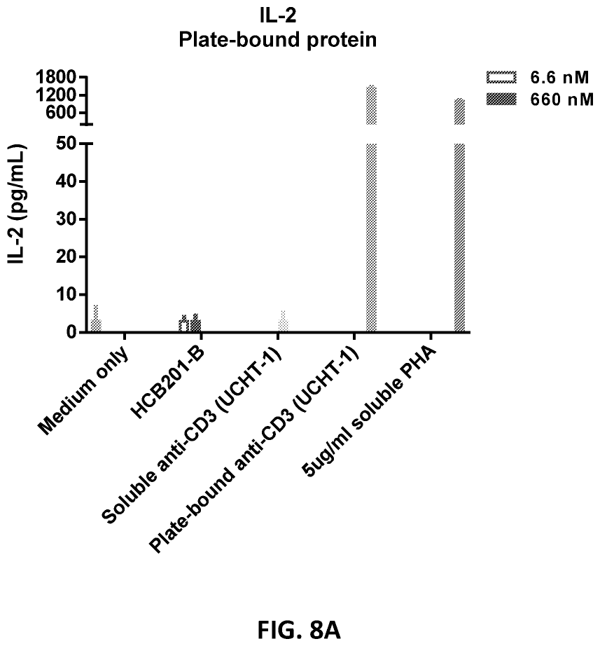

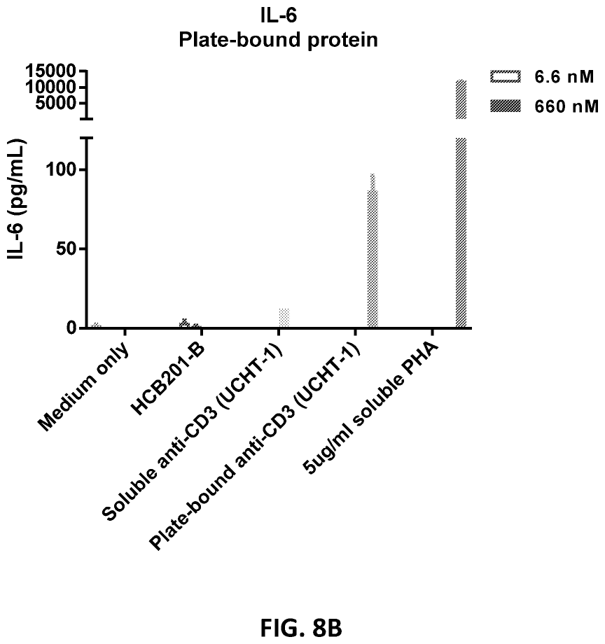

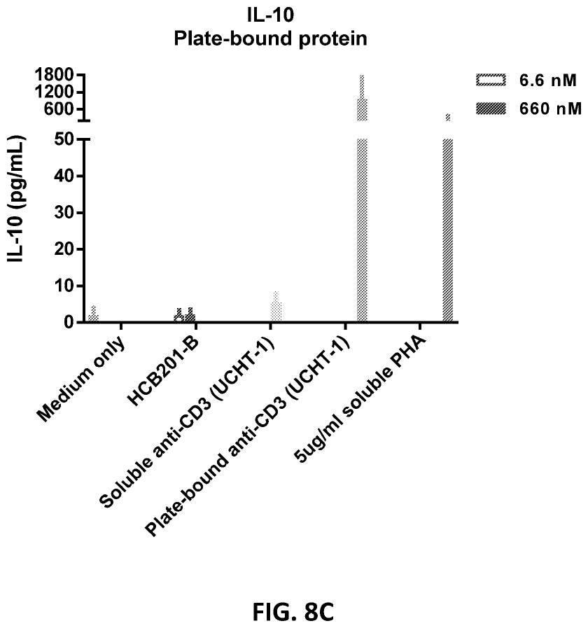

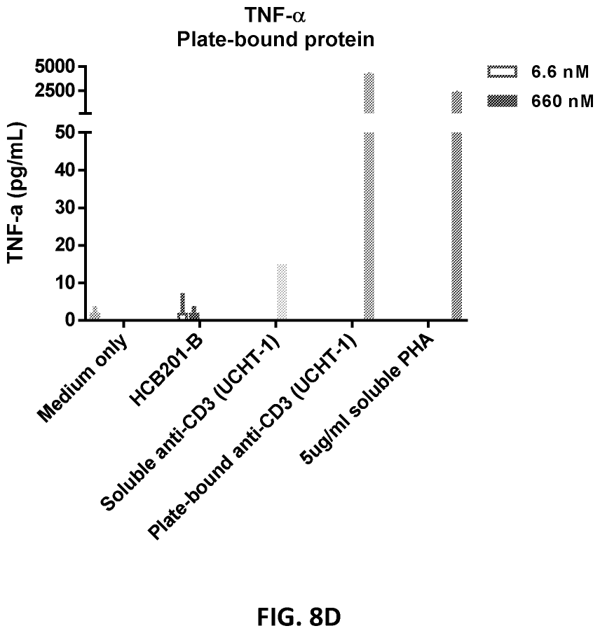

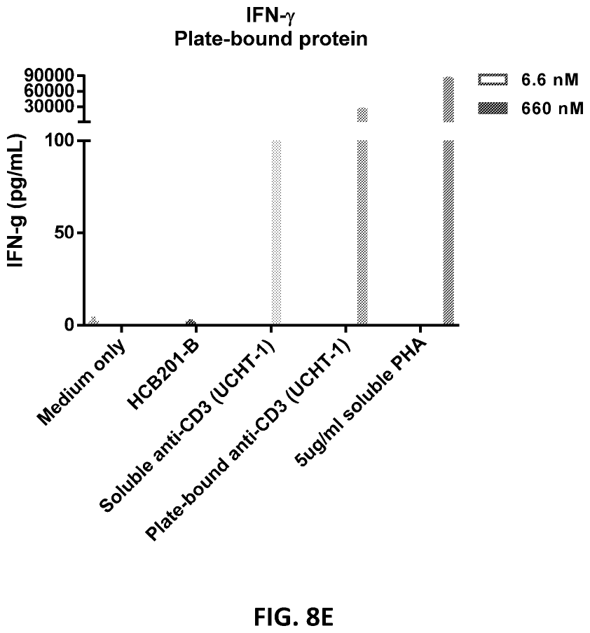

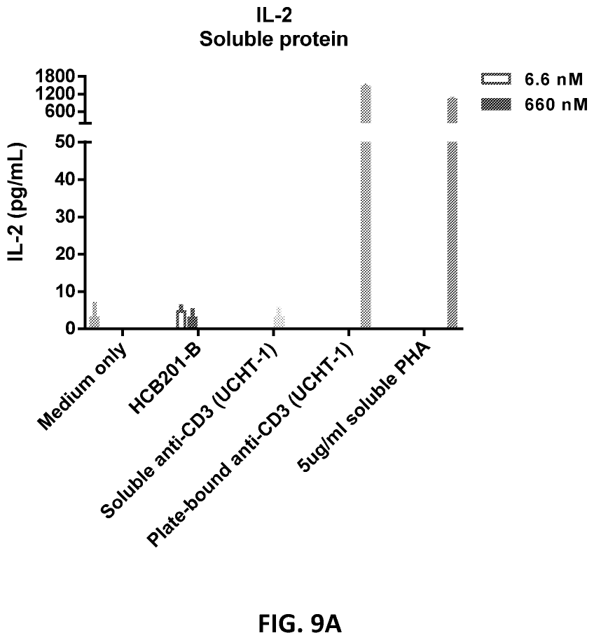

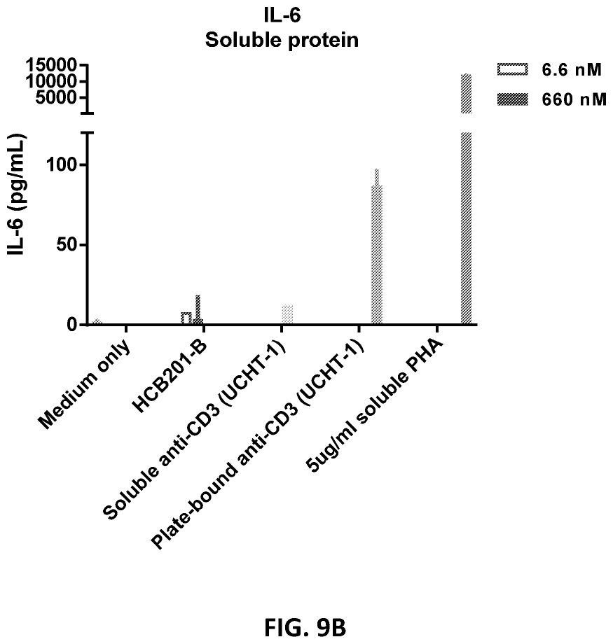

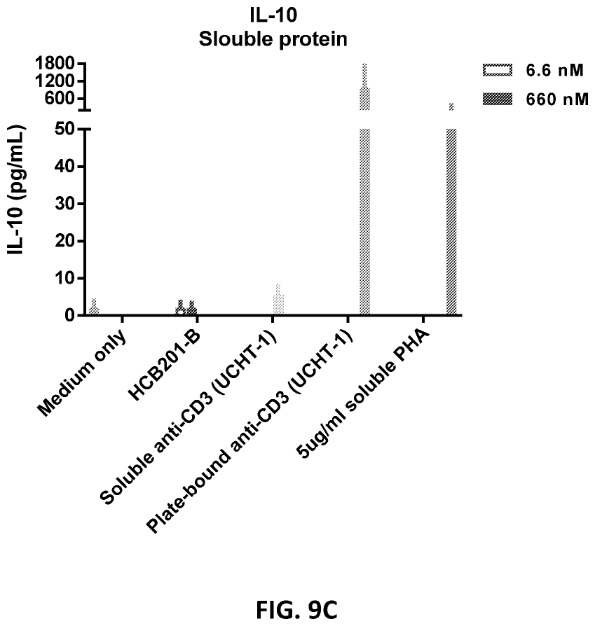

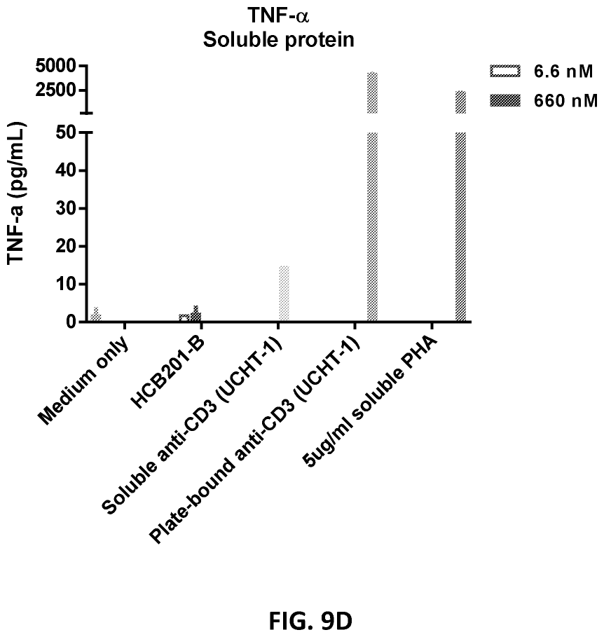

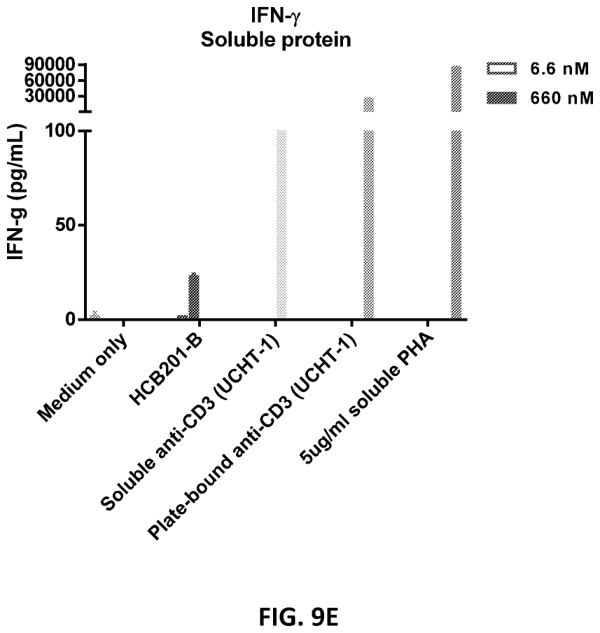

As shown in FIGS. 8A-8E, immobilized HCB201-B did not overly induce cytokine release from human PBMCs as compared to the positive controls. As shown in FIGS. 9A-9E, soluble HCB201-B did not overly induce cytokine release from human PBMCs as compared to the negative controls. The above results indicate that HCB201-B is not a superagonist and has low risk to induce the cytokine storm in human.

### Example 9. Determination of Inflammatory Cytokine Release from Macrophages

Previous reports have shown that urelumab (BMS-663513), a fully human IgG4 monoclonal antibody developed by Bristol-Myers Squibb, induced inflammatory hepatotoxicity. As disclosed in Eskiocak, U. et al. “Differentiated agonistic antibody targeting CD137 eradicates large tumors without hepatotoxicity.” JCI insight 5.5 (2020), primary human monocyte-derived macrophages (MDM) were stimulated with 10 μg/ml of CpG ODN-SL01 (double-stem loop ODN), which is a synthetic oligonucleotide that contains unmethylated CpG dinucleotides in particular sequence contexts (CpG motifs). The stimulated MDM cells were recognized by TLR9 leading to strong immunostimulatory effects. The effects were further aggravated by urelumab after an incubation of 48 hours, as demonstrated by increased TNF-α and IL-27 secretion levels.

Because of the inflammatory hepatotoxicity that can be induced by anti-4-1BB antibody urelumab, experiments were designed to assess inflammatory cytokine TNF-α and IL-27 secretion from macrophages. Specifically, 1×10⁴ cells/well of MDM cells were seeded in a 96-well plate and then stimulated by 1 μg/ml of LPS (lipopolysaccharide). The MDM cells were then incubated with antibodies and HCB201 proteins at indicated concentrations for 48 hours. The culture supernatants were collected for determination of TNF-α and IL-27 levels by Human TNF-α ELISA MAXIM Deluxe kit (BioLegend) and Human IL-27 DuoSet ELISA kit (R&D system), respectively. Urelumab was used as a positive control. Anti-4-1BB antibody utomilumab, a fully human IgG2 monoclonal antibody, was used as a negative control.

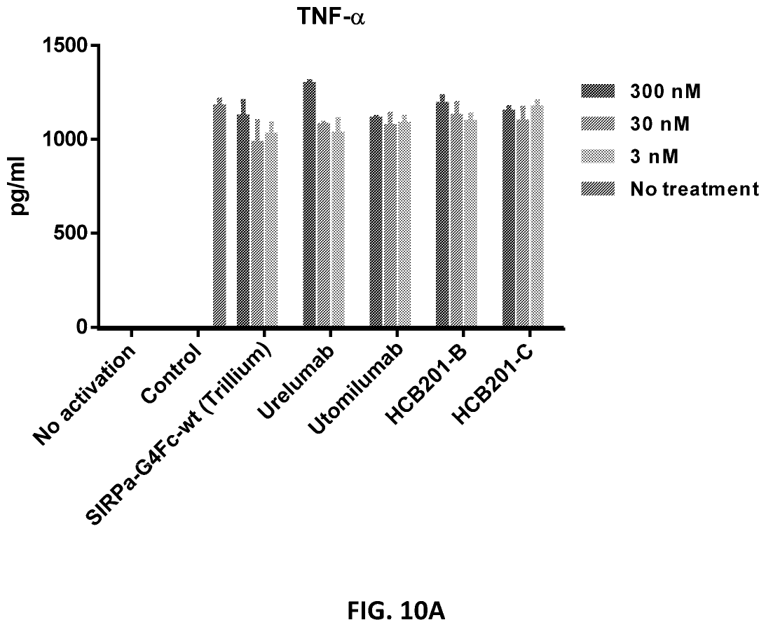

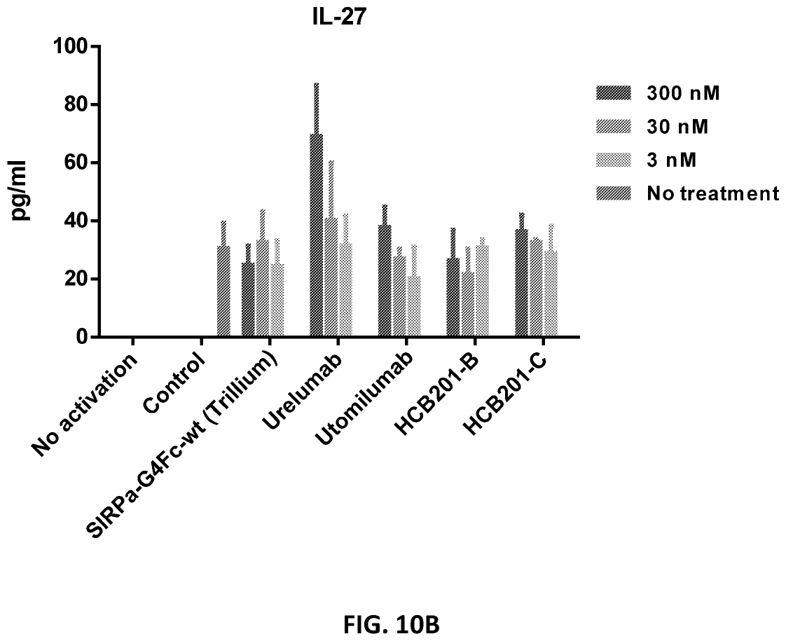

As shown in FIGS. 10A-10B, HCB201-B did not enhance the secretion of TNF-α and IL-27 as compared to urelumab. This result indicates that HCB201-B has a low risk on the induction of hepatotoxicity.

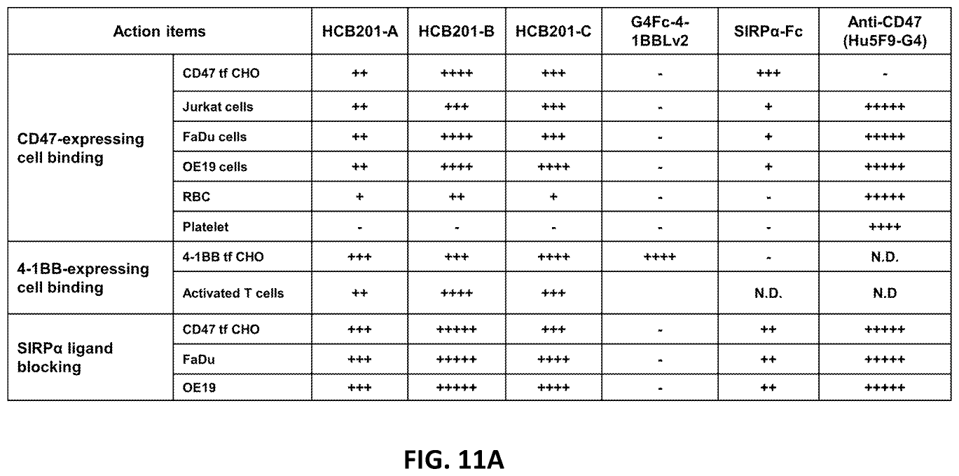

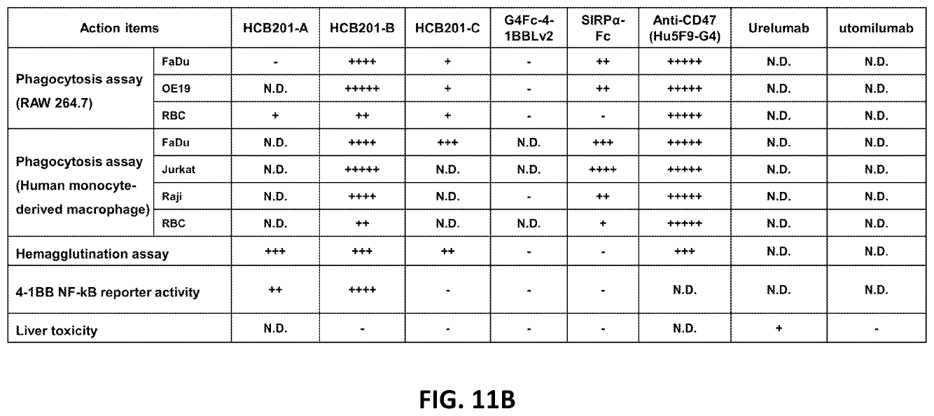

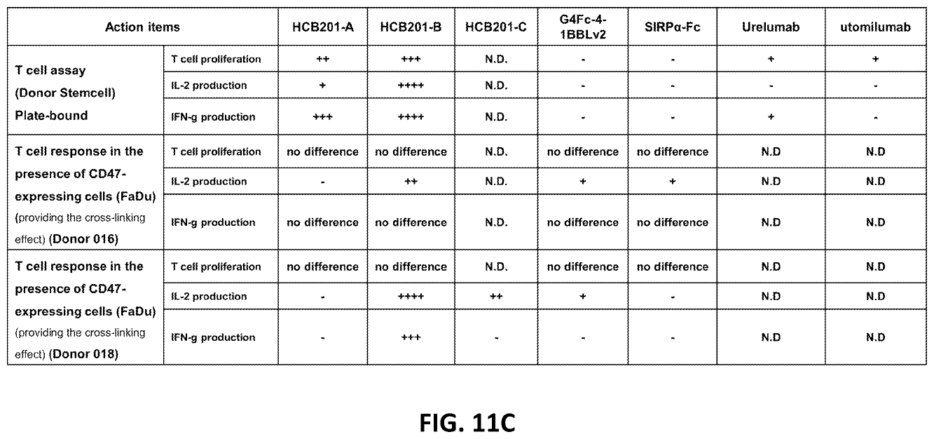

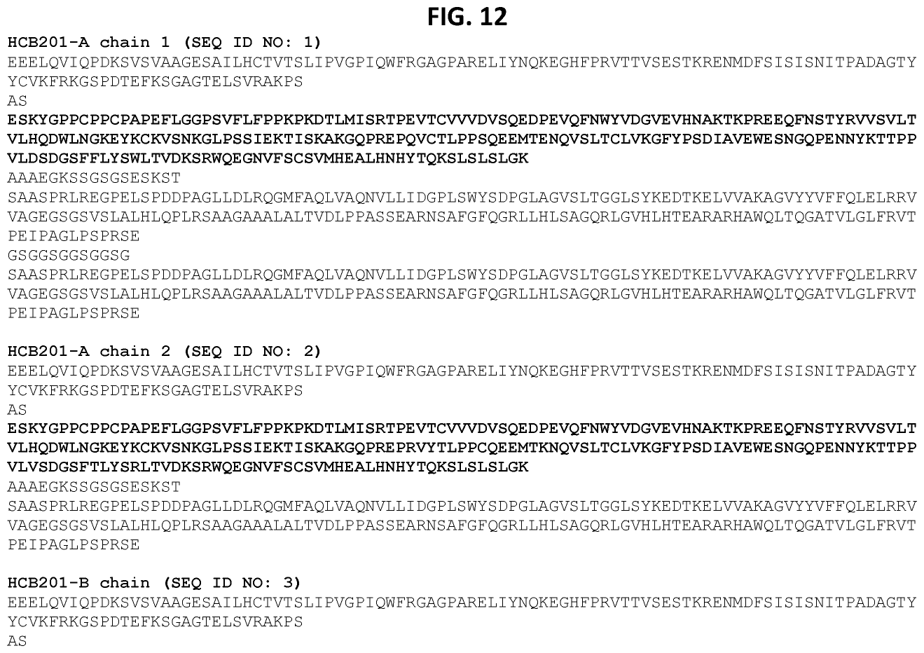

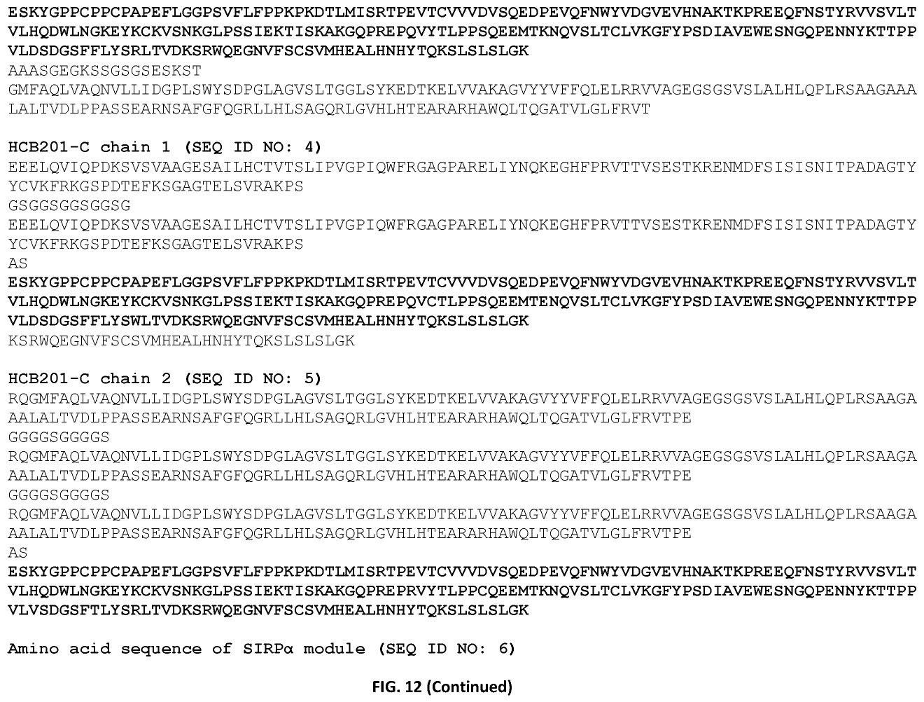

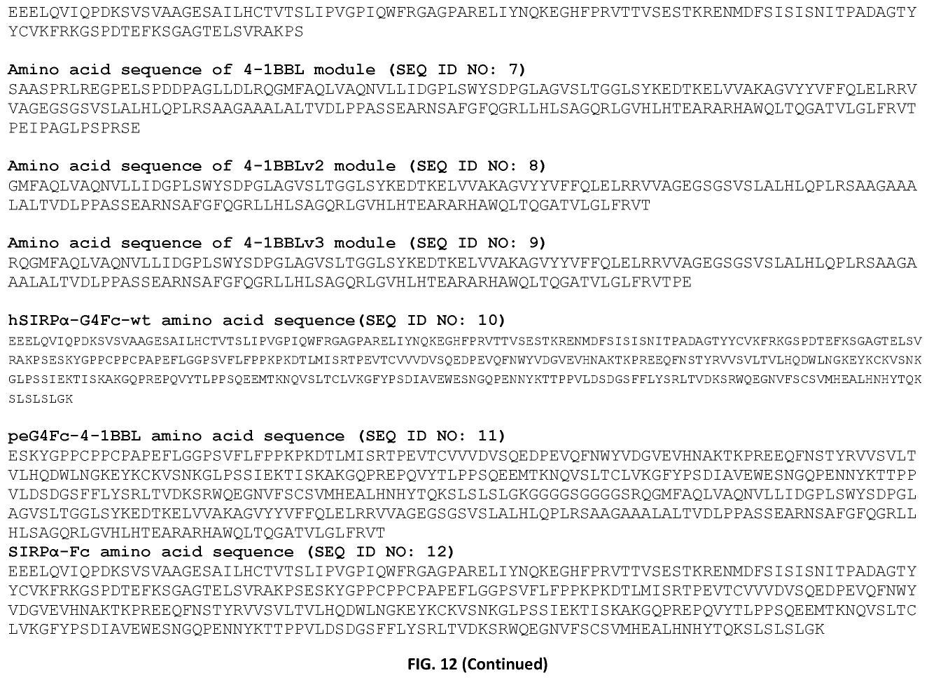

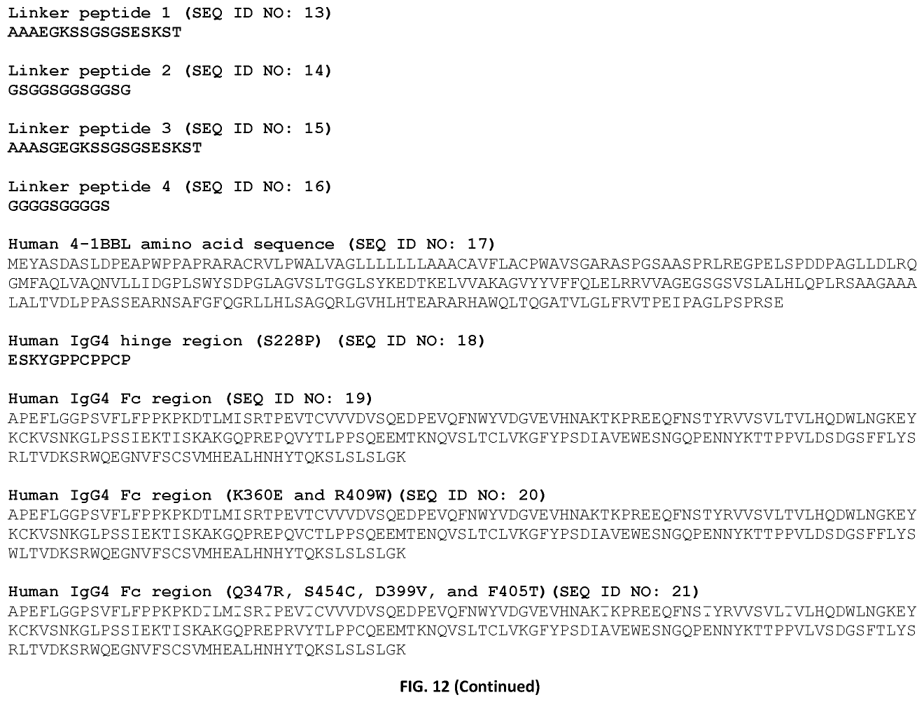

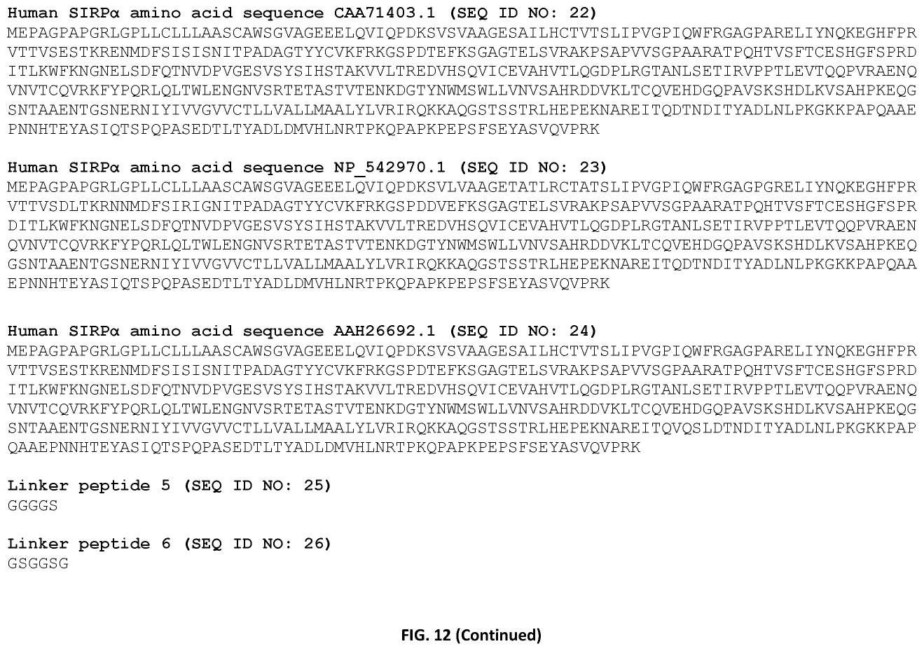

As a summary, FIGS. 11A-11C show the in vitro assay results as discussed above. Compared to other HCB201 proteins or control antibodies, HCB201-B has a greater CD47-expressing cell binding and SIRPα ligand blocking activity. Furthermore, HCB201B showed a similar ability to induce macrophage-mediated phagocytosis on tumor cells and the lower risk to induce macrophage-mediated phagocytosis on human RBC, as compared to anti-CD47 antibody Hu5F9. 4-1BB reporter assays and T cell-based assays also demonstrated that HCB201-B could significantly induce activation of 4-1BB-expressing cells, T cell proliferation and cytokine release. All these data suggested that HCB201-B has superior properties as compared to HCB201-A, HCB201-C, and control antibodies or proteins.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A protein complex, comprising:
(a) an Fc;
(b) a CD47-binding domain; and
(c) a 4-1BB (tumor necrosis factor receptor superfamily member 9)-binding domain.

2. The protein complex of claim 1, wherein the CD47-binding domain can bind to a cell (e.g., cancer cell) expressing CD47 and/or block the interaction between CD47 and signal regulatory protein α (SIRPα).

3. The protein complex of claim 1 or 2, wherein the CD47-binding domain is or comprises a SIRPα extracellular domain.

4. The protein complex of claim 3, wherein the SIRPα extracellular domain is a human SIRPα extracellular domain.

5. The protein complex of claim 4, wherein the CD47-binding domain comprises an amino acid sequence that is at least 80%, 85%, 90%, 95%, 96%, 97%, 98%, 99% or 100% identical to SEQ ID NO: 6.

6. The protein complex of any one of claims 1-5, wherein the 4-1BB-binding domain can bind to an immune cell (e.g., T cell) expressing 4-1BB and/or stimulate T cell activation and proliferation.

7. The protein complex of any one of claims 1-6, wherein the 4-1BB-binding domain is or comprises a 4-1BBL extracellular domain.

8. The protein complex of claim 7, wherein the 4-1BBL extracellular domain is a human 4-1BBL extracellular domain.

9. The protein complex of claim 8, wherein the 4-1BB-binding domain comprises an amino acid sequence that is at least 80%, 85%, 90%, 95%, 96%, 97%, 98%, 99% or 100% identical to SEQ ID NO: 7, 8, or 9.

10. The protein complex of any one of claims 1-9, wherein the Fc is human IgG4 Fc.

11. The protein complex of any one of claim 1-10, wherein the CD47-binding domain is linked to the N-terminus of a CH2 domain in the Fc, optionally via a hinge region.

12. The protein complex of any one of claims 1-11, wherein the 4-1BB-binding domain is linked to the N-terminus of a CH2 domain in the Fc, optionally via a hinge region.

13. The protein complex of claim 11 or 12, wherein the hinge region is a human IgG4 hinge region optionally with S228P mutation according to EU numbering.

14. The protein complex of any one of claims 1-11, wherein the 4-1BB-binding domain is linked to the C-terminus of a CH3 domain in the Fc, optionally via a linker peptide.

15. The protein complex of any one of claims 1-14, wherein the Fc comprises a first CH3 domain and a second CH3 domain, wherein either one or both CH3 domain comprise one or more amino acid residues selected from 360E, 409W, 347R, 399V, and 405T (EU numbering).

16. The protein complex of any one of claims 1-15, wherein the protein complex comprises two or more CD47-binding domains.

17. The protein complex of any one of claims 1-16, wherein the protein complex comprises two or more 4-1BB-binding domains.

18. A protein complex, comprising
(a) a first polypeptide comprising from N-terminus to C-terminus: a first CD47-binding domain, an optional first hinge region, a first Fc region, an optional first linker peptide, a first 4-1BB-binding domain, and a second 4-1BB-binding domain; and
(b) a second polypeptide comprising from N-terminus to C-terminus: a second CD47-binding domain, an optional second hinge region, a second Fc region, an optional second linker peptide, and a third 4-1BB-binding domain.

19. The protein complex of claim 18, wherein the first CD47-binding domain and/or the second CD47-binding domain comprise a sequence that is at least 80%, 90%, 95%, or 100% identical to SEQ ID NO: 6.

20. The protein complex of claim 18 or 19, wherein the first 4-1BB-binding domain, the second 4-1BB binding domain, and/or the third 4-1BB-binding domain comprise a sequence that is at least 80%, 90%, 95%, or 100% identical to SEQ ID NO: 7.

21. The protein complex of any one of claims 18-20, wherein the first hinge region and/or the second hinge region comprise a sequence that is at least 80% identical to SEQ ID NO: 18.

22. The protein complex of any one of claims 18-21, wherein the first Fc region comprises a sequence that is at least 80% identical to SEQ ID NO: 20, and the second Fc region comprises a sequence that is at least 80%, 90%, 95%, or 100% identical to SEQ ID NO: 21.

23. The protein complex of any one of claims 18-22, wherein the first linker peptide and/or the second linker peptide comprise a sequence that is at least 80% identical to SEQ ID NO: 13.

24. The protein complex of any one of claims 18-23, wherein the first 4-1BB-binding domain and the second 4-1BB-binding domain are linked via a third linker peptide, wherein the third linker peptide comprises a sequence that is at least 80% identical to SEQ ID NO: 14.

25. The protein complex of any one of claims 18-24, wherein the first polypeptide comprises a sequence that is at least 80%, 90%, 95%, or 100% identical to SEQ ID NO: 1, and the second polypeptide comprises a sequence that is at least 80%, 90%, 95%, or 100% identical to SEQ ID NO: 2.

26. A protein complex, comprising
(a) a first polypeptide comprising from N-terminus to C-terminus: a first CD47-binding domain, an optional first hinge region, a first Fc region, an optional first linker peptide, and a first 4-1BB-binding domain; and
(b) a second polypeptide comprising from N-terminus to C-terminus: a second CD47-binding domain, an optional second hinge region, a second Fc region, an optional second linker peptide, and a second 4-1BB-binding domain.

27. The protein complex of claim 26, wherein the first CD47-binding domain and/or the second CD47-binding domain comprise a sequence that is at least 80%, 90%, 95%, or 100% identical to SEQ ID NO: 6.

28. The protein complex of claim 26 or 27, wherein the first 4-1BB-binding domain and/or the second 4-1BB-binding domain comprise a sequence that is at least 80%, 90%, 95%, or 100% identical to SEQ ID NO: 8.

29. The protein complex of any one of claims 26-28, wherein the first hinge region and/or the second hinge region comprise a sequence that is at least 80% identical to SEQ ID NO: 18.

30. The protein complex of any one of claims 26-29, wherein the first Fc region and the second Fc region comprise a sequence that is at least 80%, 90%, 95%, or 100% identical to SEQ ID NO: 19.

31. The protein complex of any one of claims 26-30, wherein the first linker peptide and/or the second linker peptide comprise a sequence that is at least 80% identical to SEQ ID NO: 15.

32. The protein complex of any one of claims 26-31, wherein the first polypeptide comprises a sequence that is at least 80%, 90%, 95%, or 100% identical to SEQ ID NO: 3, and the second polypeptide comprises a sequence that is at least 80%, 90%, 95%, or 100% identical to SEQ ID NO: 3.

33. A protein complex, comprising
(a) a first polypeptide comprising from N-terminus to C-terminus: a first CD47-binding domain, a second CD47-binding domain, an optional first hinge region, and a first Fc region; and
(b) a second polypeptide comprising from N-terminus to C-terminus: a first 4-1BB-binding domain, a second 4-1BB-binding domain, a third 4-1BB-binding domain, an optional second hinge region, and a second Fc region.

34. The protein complex of claim 33, wherein the first CD47-binding domain and/or the second CD47-binding domain comprise a sequence that is at least 80%, 90%, 95%, or 100% identical to SEQ ID NO: 6.

35. The protein complex of claim 33 or 34, wherein the first 4-1BB-binding domain, the second 4-1BB binding domain, and/or the third 4-1BB-binding domain comprise a sequence that is at least 80%, 90%, 95%, or 100% identical to SEQ ID NO: 9.

36. The protein complex of any one of claims 33-35, wherein the first hinge region and/or the second hinge region comprise a sequence that is at least 80% identical to SEQ ID NO: 18.

37. The protein complex of any one of claims 33-36, wherein the first Fc region comprises a sequence that is at least 80% identical to SEQ ID NO: 20, and the second Fc region comprises a sequence that is at least 80%, 90%, 95%, or 100% identical to SEQ ID NO: 21.

38. The protein complex of any one of claims 33-37, wherein the first CD47-binding domain and the second 4-1BB-binding domain are linked via a first linker peptide, wherein the first linker peptide comprises a sequence that is at least 80% identical to SEQ ID NO: 14.

39. The protein complex of any one of claims 33-38, wherein the first 4-1BB-binding domain and the second 4-1BB-binding domain are linked via a second linker peptide, and the second 4-1BB-binding domain and the third 4-1BB-binding domain are linked via a third linker peptide, wherein the second linker peptide and/or the third linker peptide comprise a sequence that is at least 80% identical to SEQ ID NO: 16.

40. The protein complex of any one of claims 33-39, wherein the first polypeptide comprises a sequence that is at least 80%, 90%, 95%, or 100% identical to SEQ ID NO: 4, and the second polypeptide comprises a sequence that is at least 80%, 90%, 95%, or 100% identical to SEQ ID NO: 5.

41. A nucleic acid comprising a polynucleotide encoding the protein complex of any one of claims 1-40.

42. The nucleic acid of claim 41, wherein the nucleic acid is a DNA (e.g., cDNA) or RNA (e.g., mRNA).

43. A vector comprising one or more of the nucleic acids of claim 41 or 42.

44. A cell comprising the vector of claim 43.

45. The cell of claim 44, wherein the cell is a CHO cell.

46. A cell comprising one or more of the nucleic acids of claim 41 or 42.

47. A method of producing a protein complex, the method comprising
(a) culturing the cell of any one of claims 44-46 under conditions sufficient for the cell to produce the protein complex; and
(b) collecting the protein complex produced by the cell.

48. An antibody-drug conjugate comprising the protein complex of any one of claims 1-40, covalently bound to a therapeutic agent.

49. The antibody drug conjugate of claim 48, wherein the therapeutic agent is a cytotoxic or cytostatic agent.

50. A method of treating a subject having cancer, the method comprising administering a therapeutically effective amount of a composition comprising the protein complex of any one of claims 1-40, or the antibody-drug conjugate of claim 48 or 49, to the subject.

51. The method of claim 50, wherein the subject has a cancer cell expressing CD47.

52. The method of claim 50 or 51, wherein the cancer is ovarian cancer, head and neck squamous cell cancer, melanoma, non-small cell lung cancer, leukemia, breast cancer, small-cell lung cancer, lymphoma, or colorectal cancer.

53. A method of decreasing the rate of tumor growth, the method comprising contacting a tumor cell with an effective amount of a composition comprising the protein complex of any one of claims 1-40, or the antibody-drug conjugate of claim 48 or 49.

54. A method of killing a tumor cell, the method comprising contacting a tumor cell with an effective amount of a composition comprising the protein complex of any one of claims 1-40, or the antibody-drug conjugate of claim 48 or 49.

55. A pharmaceutical composition comprising the protein complex of any one of claims 1-40, and a pharmaceutically acceptable carrier.
